(19) United States
(12) Patent Application Publication
(10) Pub. No.: US 2009/0221787 A1
(43) Pub. Date: Sep. 3, 2009

(54) Production Of Monomer, Oligomer And Polymer Phosphonic Acid Esters And Phosphonic And Sulphonic Acids By A Nucleophile Aromatic Substitution

(76) Inventors: Thomas Haring, Stuttgart (DE); Jochen Kerres, Ostfidern (DE); Martin Hein, Basel (CH); Frank Schonberger, Stuttgart (DE)

Correspondence Address: FITCH EVEN TABIN & FLANNERY, 120 SOUTH LASALLE STREET, SUITE 1600, CHICAGO, IL, 60603-3406, US

(21) Appl. No.: 11/995,187

(22) PCT Filed: Jul. 10, 2006
(86) PCT No.: PCT/DE06/01240

§ 371 Date: Oct. 2, 2008

(30) Foreign Application Priority Data
Jul. 9, 2005 (DE) 10 2005 032 198.4
Aug. 20, 2005 (DE) 10 2005 040 583.5
Mar. 31, 2006 (DE) 10 2006 016 258.7

(51) Int. Cl. C08G 75/00; C07C 315/00; C07F 9/02; C08G 73/24

(52) U.S. Cl. 528/391; 568/28; 568/14; 528/401

(57) ABSTRACT

The invention relates to producing monomer, oligomer and polymer non-fluorinated, partially fluorinated or perfluorinated sulphonic acids by reacting halogenated, low-molecular weight, oligomer or macromolecular arenes with (hydrogen) sulphites, dithionites, sulphides or other reducing sulfur salts, possibly by oxidising sulphur-containing arene intermediates which are formed at a sulphur oxidation degree less than +6 by means of appropriate oxidation agents with formation of corresponding sulphonate functional groups (sulphonic acid, sulfohalogenide, sulphonamide and sulphonic acid ester groups). The invention also relates to a method for producing monomer, oligomer or polymer non-halogenated, partially halogenated or perhalogenated phosphonic acid esters and the derivatives thereof (for example, a free phosphonic acid or a phosphonic acid salt) by a nucleophile aromatic substitution (Michaelis-Becker-reaktion, complete or partial substitution of halogenide functional groups by phosphate functional groups (phosphonic acid esters, phosphonic acid amides, phosphonic acids, phosphonic acid salts, phosphonic acid halogenides). Said invention also relates to a method for producing polymer or ionomer non-halogenated, partially halogenated or perhalogenated containing (CF₂)ₓPO(OR)₂— or (CF₂)ₓSO₂Me- or (CF₂)ₓSO₃Me- side chains, (wherein x=1-20, R=any organic radical, Me=any monovalent cation). A method for carrying out a nucleophile polycondensation of said functionalised (i.e. sulphonated, sulphonated or phosphonated) monomers into oligomers or polymers is also disclosed.

### SUMMARY

What is claimed is:
Process to produce monomeric, oligomeric and polymeric non-, partial- or perfluorinated sulfonic acids by reaction of halogenated low molecular, oligomeric or macro-molecular arenes with (hydrogen)sulfites, dithionites, sulfides or other reducing sulphur salts, if necessary by oxidation of formed sulphur containing arene intermediate compounds with a lower oxidation state of the sulphur than +6 with suitable oxidation reagents and formation of the corresponding sulfonated functional groups (sulfonic acid groups, sulfohalogenide groups, sulphonamide groups, sulfonic acid ester groups).
Process to produce monomeric, oligomeric and polymeric non-, partial- or perhalogenated phosphonic acid ester as well as their derivatives (e.g. free phosphonic acid or salt of phosphonic acid) by nucleophilic aromatic substitution (Michaelis-Becker-reaction, in part or complete substitution of the halogenide functional groups by phosphonate functional groups (phosphonic acid ester, phosphonic acid amides, phosphonic acids, phosphonic acid salts, phosphonic acid halogenides)
Process to produce non-, partial- or perfluorinated, (CF₂)ₓPO(OR)₂— or (CF₂)ₓSO₂Me- or (CF₂)ₓSO₃Me side chains (x=1-20, R=any organic radical, Me=any monovalent cation) containing polymers respective ionomers.
Process for nucleophilic polycondensation of the mentioned functionalised (that is sulfonated, sulfonated respective phosphonated) monomers to oligomers or polymers.

### STATE-OF-THE-ART

The usual processes to produce sulfonated poly(aryl)ether can be divided into the following two groups¹:
i.) Subsequent sulfonation of existing polymers:
In the processes described in the literature always reactions of the typ electrophilic aromatic substitution (S_{E}Ar) are used. As typical sulfonation reagents are mentionned concentrated sulphuric acid, oleum, chlorsulfonic acid, sulphur trioxide as well as their coordination compounds (e.g. sulphur trioxide-pyridine, sulphur trioxide-triethylphosphate-complex). Alternative sulfonation pathways have been described with polysulfone as an example by Kerres et al^{2,3,4}. Here the polymer is first lithiated, in a further step reacted with the electrophile sulphur dioxide and finally the obtained polymeric sulfinate is oxidised. The lithiated polymer can also be reacted with sulfurylchloride SO₂Cl₂ to polymeric sulfochloride, followed by hydrolysis of sulfochloride groups in aqueous solvent to sulfonic acid groups
ii.) Direct copolymerisation of sulfonated polymers (statistical copolymers):
The monomers used for copolymerisation are sulfonated also by the methods described above, whereby preferentially concentrated or fuming sulphuric acid is used as sulfonation reagent^{6,7}.

During recent years direct phosphonation of polyarylehters has aroused an increasing interest. However it is synthetically difficult to bind phosphonic acids covalently to a polymer chain and has been realised so far only for a few polymers⁸. Some examples are mentioned here. The phosphonation of a poly(phosphazene)-main chain was done successfully by Allcock et al. by lithiation and subsequent reaction with chlorphosphonic acid ester⁹. Also Allcock et al. have described the phosphonation of a benzylic side chain of poly(phosphazene) with sodium dimethyl-respective sodium dibutylphosphite by nucleophilic substitution (Michaelis-Becker-Reaction). Another synthetic route to phosphonated polyarylethers is the palladium catalysed phosphonation^{10,11}. In the area of low molecular compounds the Michaelis-Becker-Reaction has been used also to produce aromatic (fluorinated) phosphonic acid ester starting from different low molecular fluorinated aromates (pentafluorobenzonitrile, octafluorotoluene, hexafluorobenzene, pentafluorobenzene, pentafluoronitrobenzene, Pentafluoroanisole) with yields of 10 to 65%¹². As far as we know this reaction has not been described for partly fluorinated polymers.

Another method to introduce a (CF₂)ₓPO(OR)₂ side chain (x=1-20, R=any organic radical) into aromatic systems is well known in the medicinal respective pharmaceutical chemistry and consist in the reaction of halogenated aromates (mostly iodiated or brominated aromate) with X(CF₂)ₓPO(OR)₂ (X=halogene, mostly bromine or iodine, R=any organic radical) in presence of zinc dust and copper bromide CuBr in N,N-dimethylacetamide (DMAc)^{13,14}. Partly and perfluorinated low molecular aromates with the general formula R—CF₂X (R=non-, partly- or perfluorinated aromates, X=Br, I) can be reacted by a process described in 15 and 16 (Sulfinatodehalogenation with subsequent oxidation) into compounds of the formula R—CF₂SO₂Y (Y=Cl, OH, OMe, Me=any cation). The above mentioned reactions have not been described as far as we know for polymers.

### Aim

The aim of this invention is to provide by nucleophilic aromatic substitution (S_{N}Ar) monomeric, oligomeric or polymeric sulfonic acids respective phosphonic acids or sulfonic acids derivatives or respective phosphonic acid derivates. Thus monomeric sulfonic acids respective phosphonic acids are obtained from partly- or perhalogenated (preferentially partly- or perfluorinated) aromates, whereas the production of oligomeric and polymeric sulfonic acids or phosphonic acids is described exemplarily for partly- or perhalogenated (preferentially partly- or perfluorinated) poly(aryl)ethers. This method can be transferred surprisingly also to other suitable partly- or perhalogenated (preferentially partly- or perfluorinated) polymers. As nucleophil to produce the above mentioned sulfonic acids is used a metal sulfite respective metal hydrogensulfite, metal dithionite or metal sulfide. As during the aromatic nucleophilic substitution reaction of halogene groups from halogenated arenes with metal dithionites respective metal hydrogendithionites or metal sulfides respective hydrogene sulfides sulphur containing functional groups are formed with a valency of sulphur below +6, these functional groups are oxidised according to the invention by oxidising reagents like molecular halogene (bromine, iodine, chlorine), metal hypochlorite, potassiumpermanganate, hydrogene peroxide or other suitable oxidising agents to the corresponding desired sulfonate functional group.

For the production of analogous phosphonic acids the Michaelis-Becker-reagent (e.g. sodiumdiethylphosphite, sodiumphenylphosphite, sodiumdibutylphosphite) serves as nucleophil at reaction temperatures of −93° C. to +200° C. The leaving group is in both cases a Cₛₚ_{²}-bound halogene (preferentially fluorine). Furthermore part of the present invention is the production of functionalised non-, partly- or perfluorinated copolymers (alternating, statistical, block- and graft copolymers) by nucleophilic polycondensation using low molecular sulfonic and/or phosphonic acids (or their derivatives), which have been partly or perfluorinated by the above mentioned methods, with corresponding diphenoles, dithiophenoles or other suitable monomers (e.g. also derivatives of diphenoles, like silylether- or carbamoyl protection group^{17,18}). For this polycondensation can be used a part from the most often used method (potassiumcarbonate as base, aprotic-dipolar solvent, relativly high temperature: 80 to 200° C., if necessary using water carrying organic solvents like benzene, toluene or xylenes) preferentially (to avoid dendrimation and crosslinking of polymers) also modified, at mild conditions usable processes. Here are mentioned especially the method described by Robertson et al.¹⁹ using molecular sieve to absorp the water formed during the reaction at comparatively low temperatures as well as a recently described process with calcium hydride as base and cesium fluoride as catalyst in DMAc/benzene respective propylene carbonate^{20,21}.

### DESCRIPTION

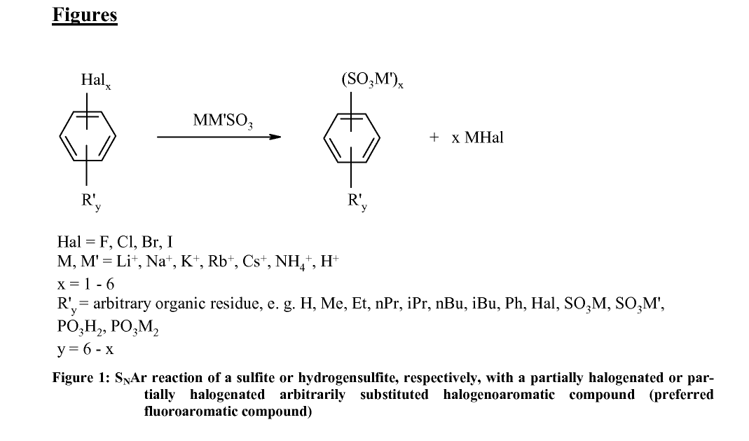

It has been found surprisingly that partly- and perhaloginated (preferentially partly- and perfluorinated) aromates according to FIG. 1 can be reacted with sulfite or hydrogenesulfite or other sulphur salts such as e.g. dithionites/hydrogenedithionites or sulfides/hydrogenesulfides in the sense of a nucleophilic aromatic substitution and provide thereby if necessary after an oxidising step the corresponding sulfonic acids or their salts.

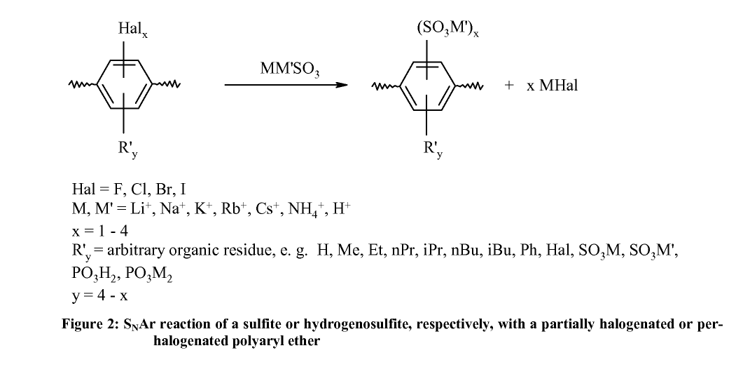

Furthermore it has been found that partly- and perhalogenated (preferentially partly- and perfluorinated) aryl main chain polymers according to FIG. 2 can be reacted with metal sulfite respective metal hydrogenesulfite in the sense of a nucleophilic aromatic substitution and provide thus polymeric sulfonic acids respective their salts.

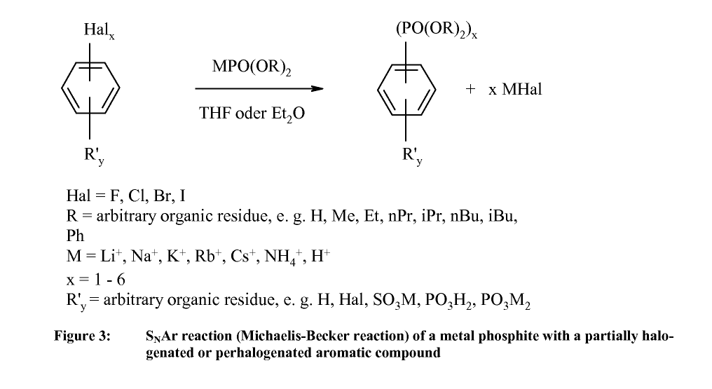

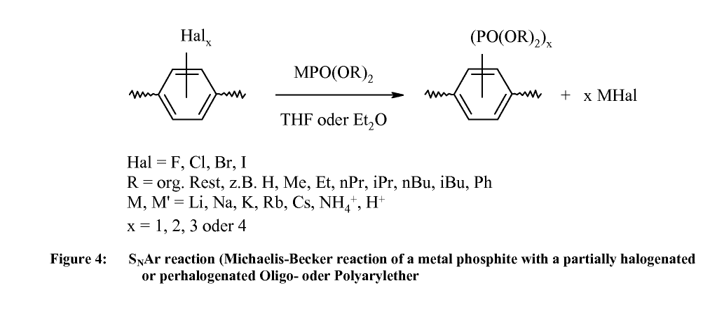

That monomeric partly- and perhalogenated aromatic compounds (see) can be reacted with metal phosphites to phosphonates (FIG. 3) (by hydrolysis of the phosphonic acid ester formed during the Michaelis-Becker-Reaction with HBr or other suitable hydrolysis reagents the free phosphonic acids are obtained) has been described for some low molecular aromatesas has been mentioned before¹². It has been found surprisingly that also part- and perhalogenated Oligo-respective Polyaryles (polyarylethers, polyarylthioethers, polyarylsulfoxides, polyarylsulfones and their copolymers) (see FIG. 4) react with metal phosphites in the sense of a Michaelis-Becker-Reaction at reaction temperatures of −93° C. to +200° C.

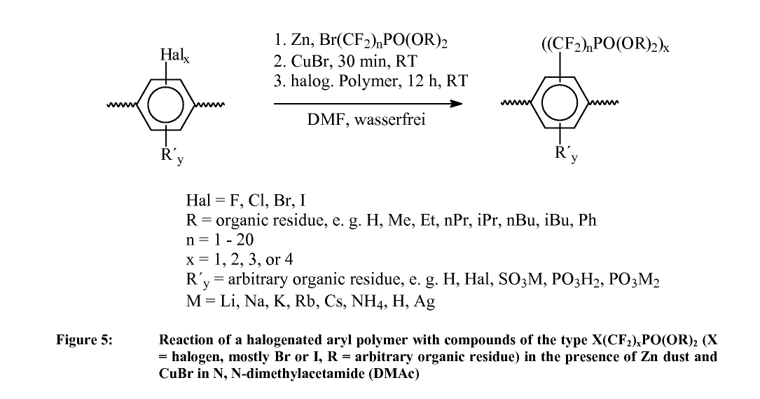

Surprisingly it has been found that the following reaction which has been described for low molecular aromates can also be done with polymers (FIG. 5).

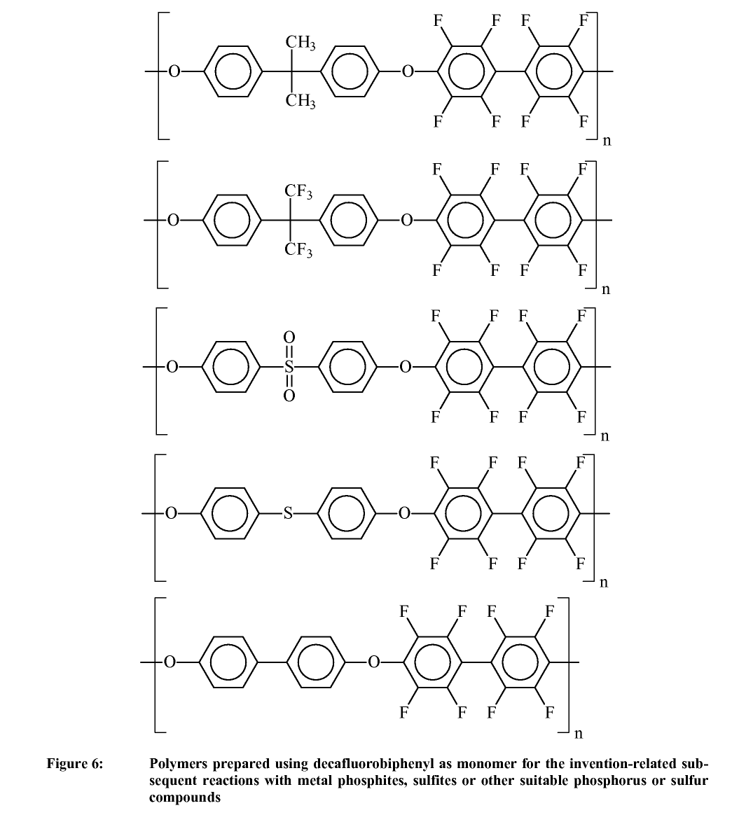

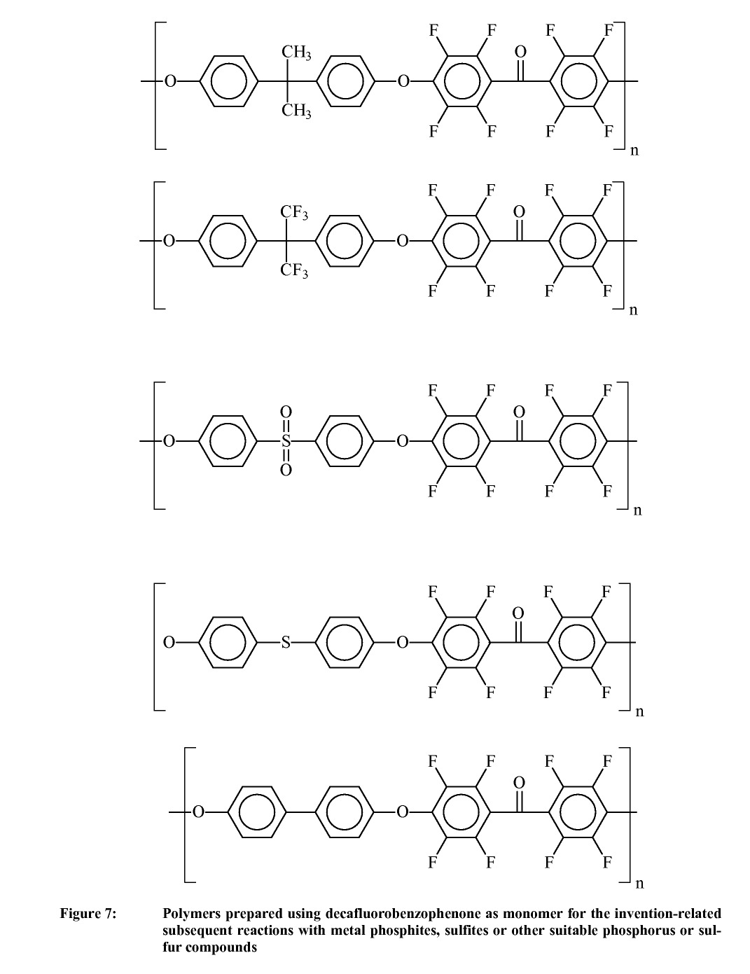

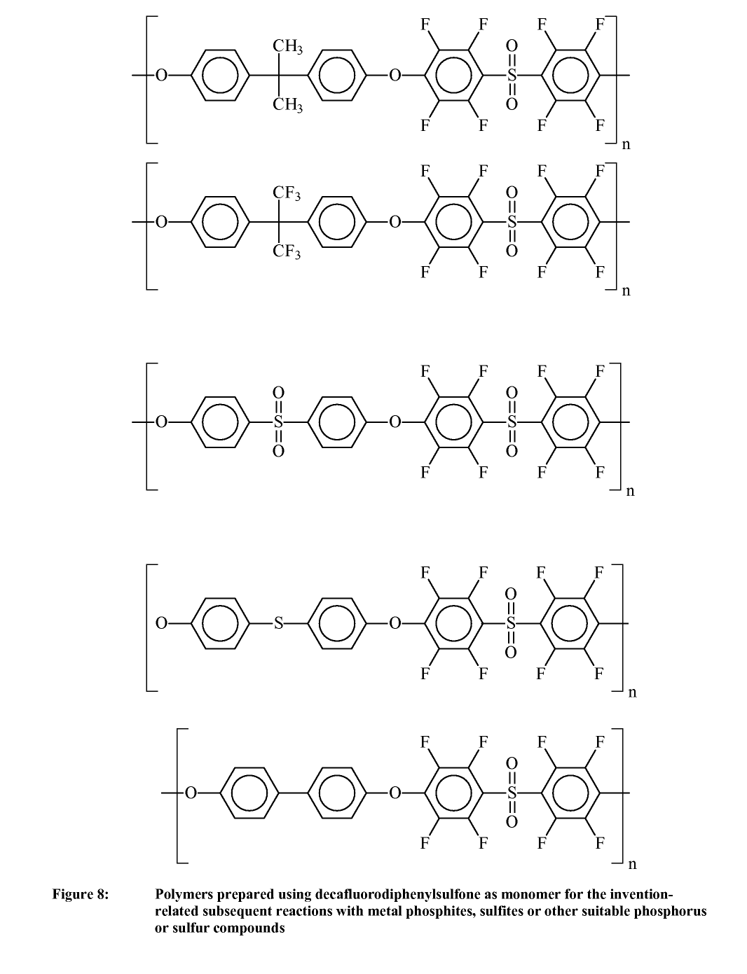

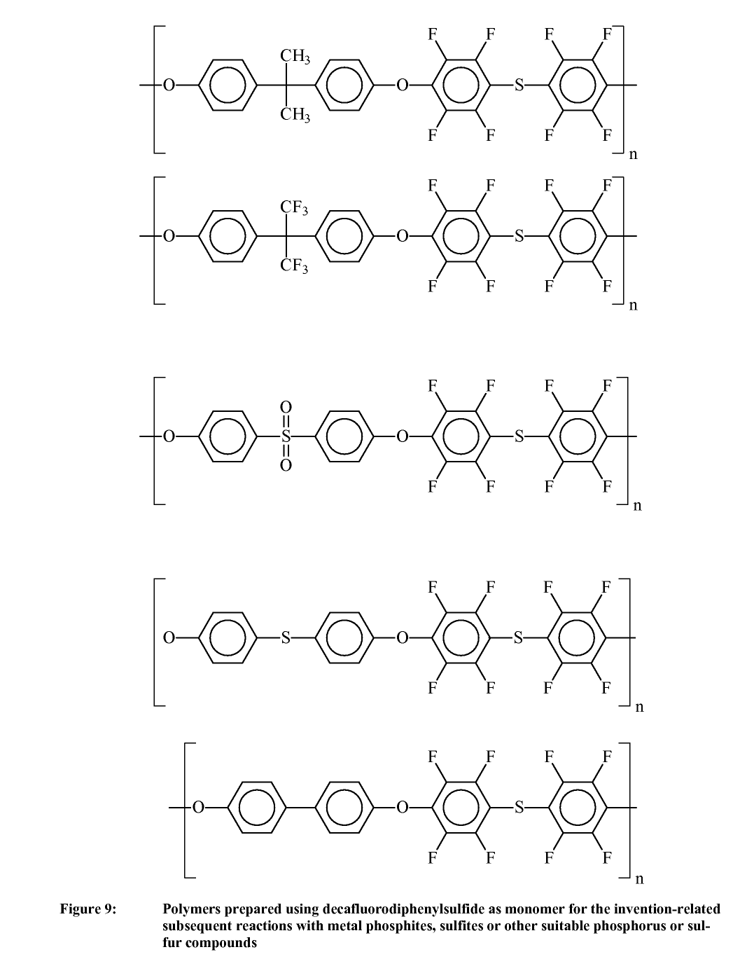

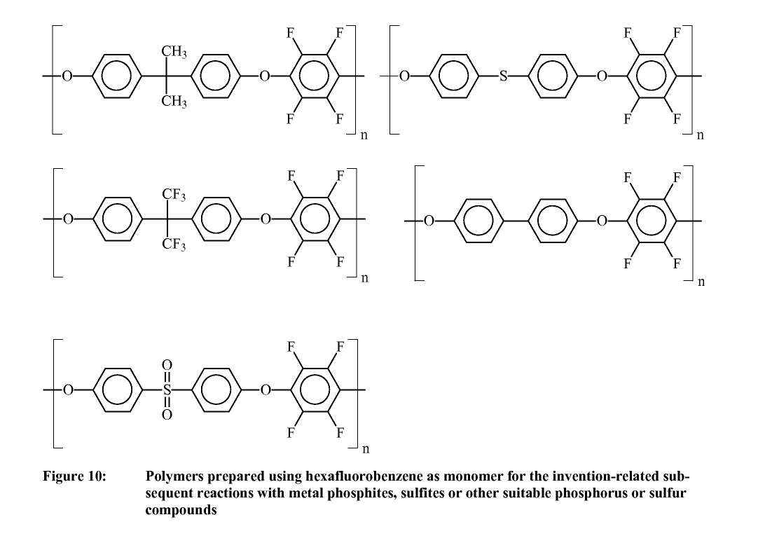

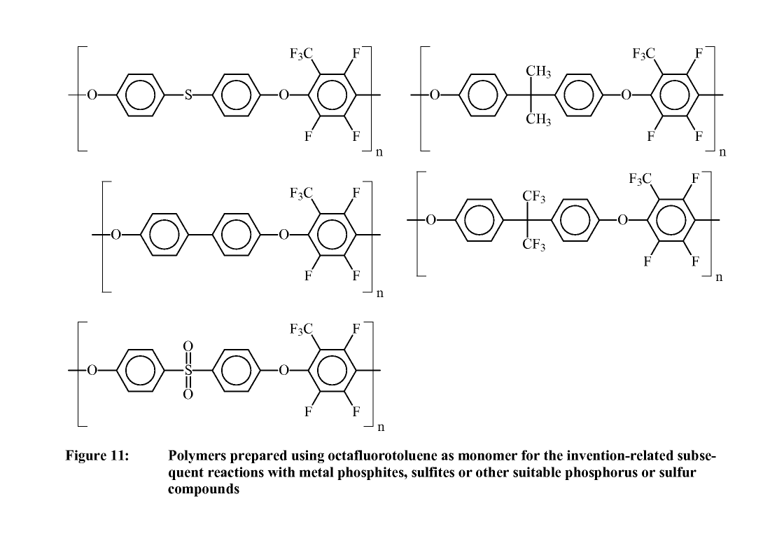

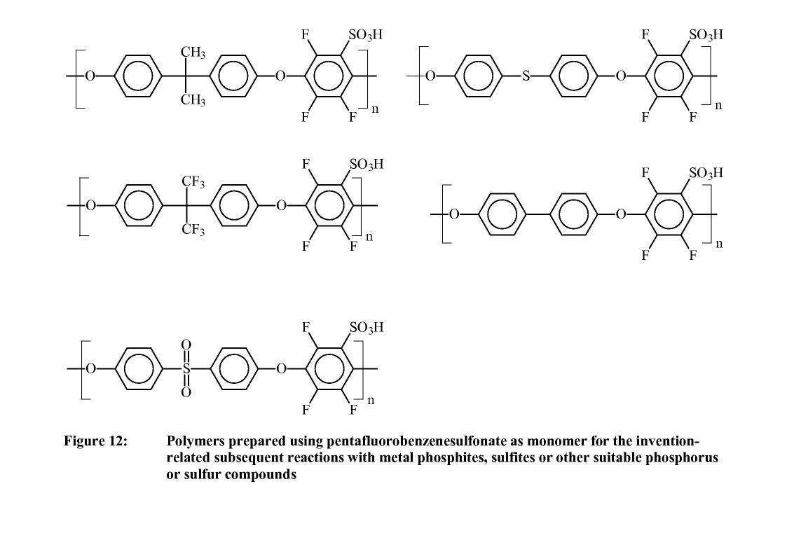

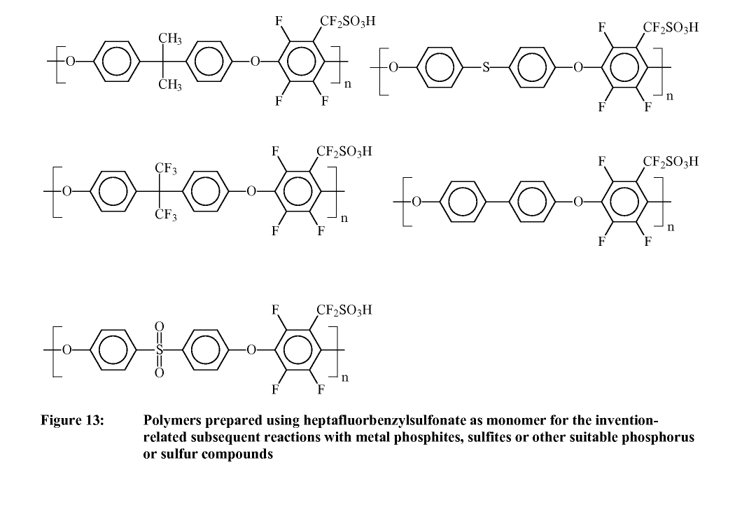

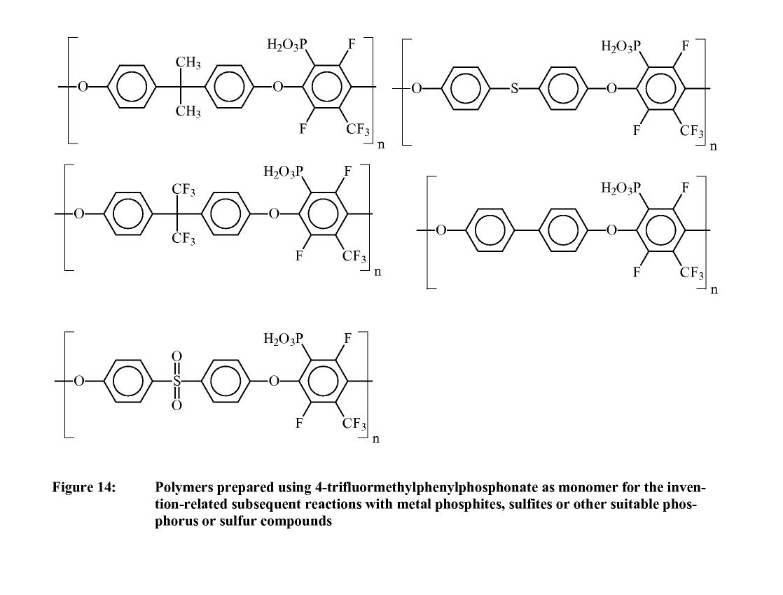

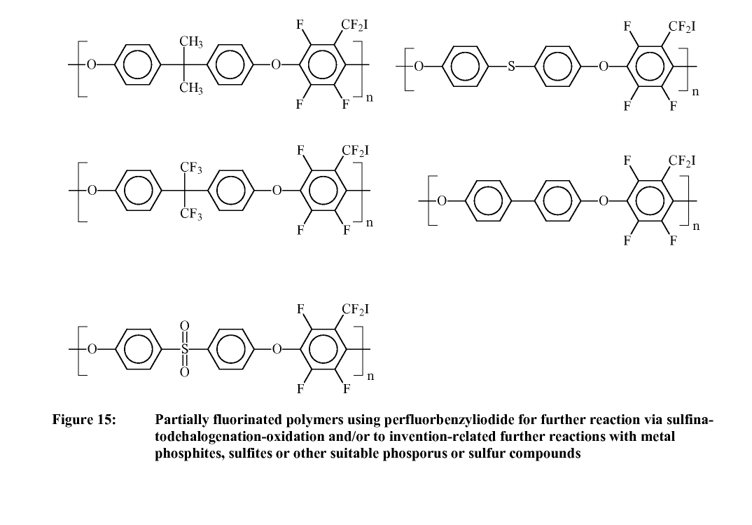

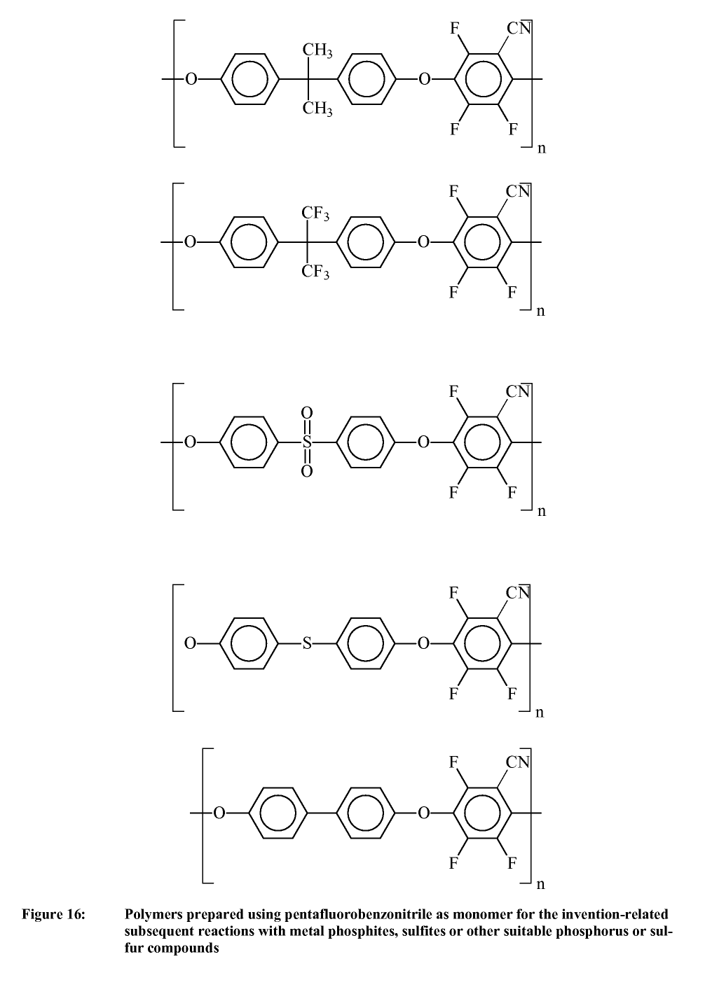

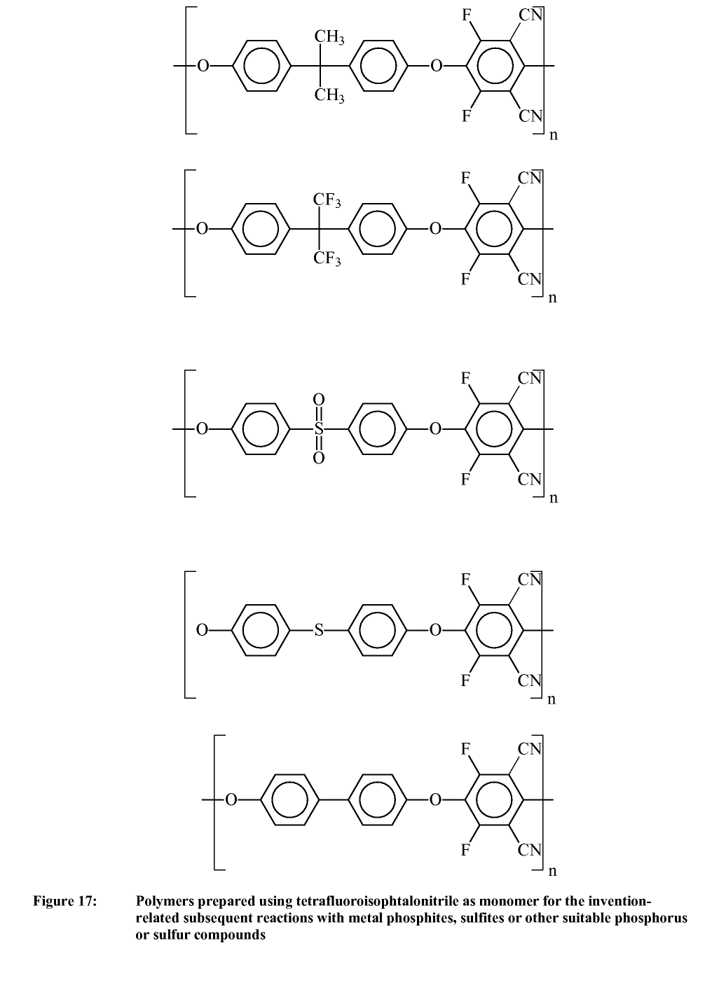

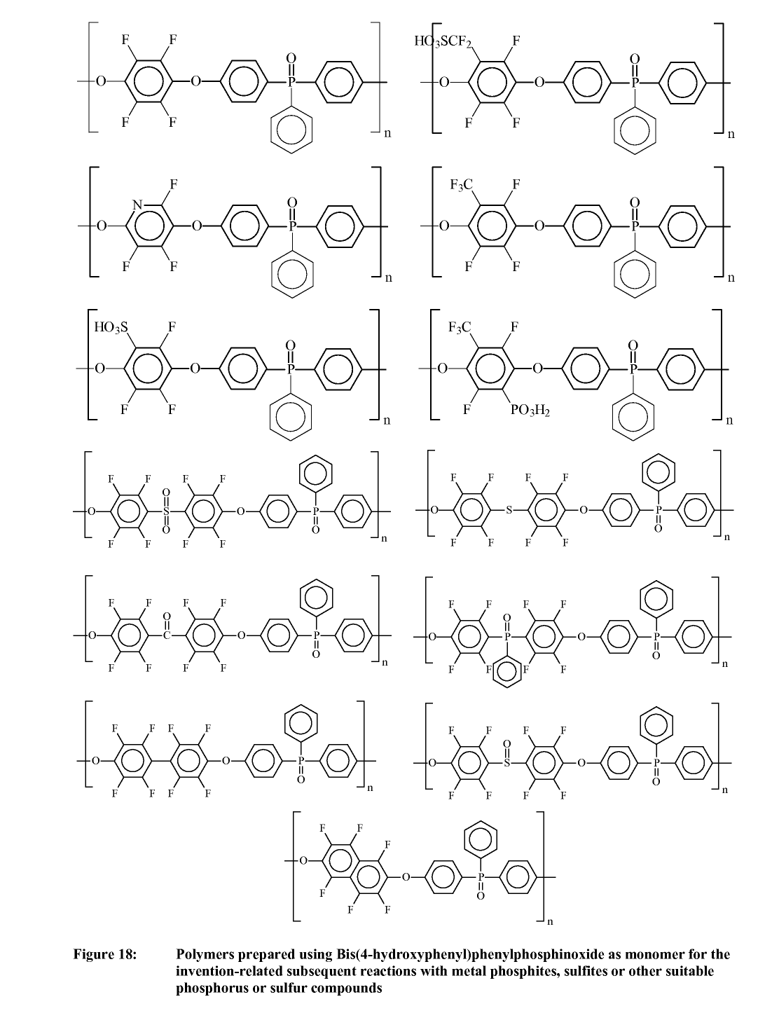

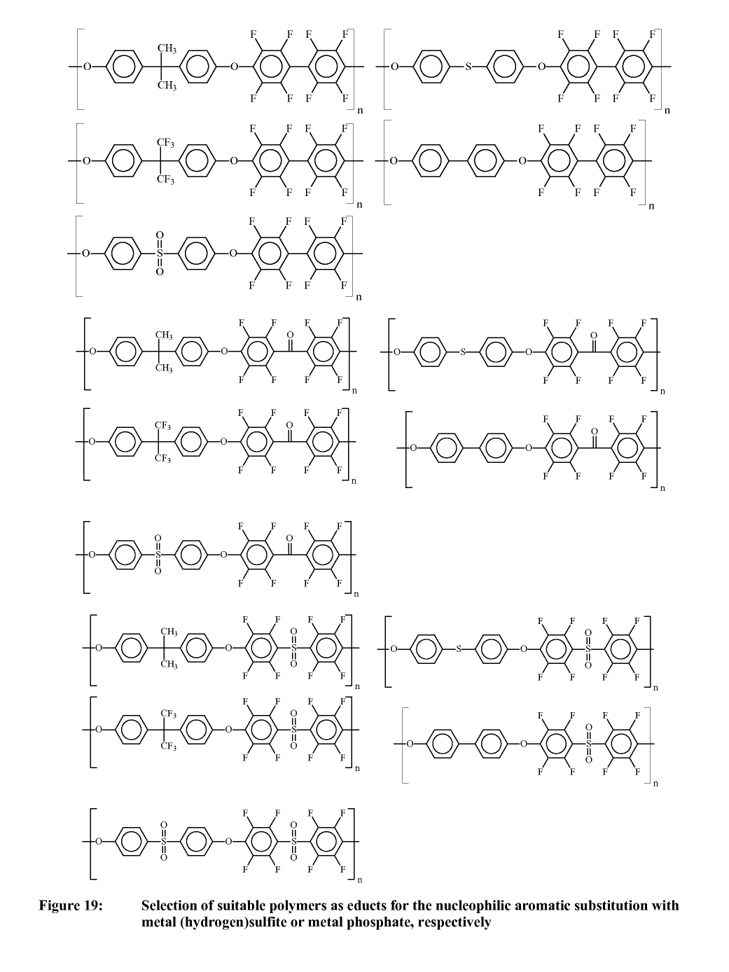

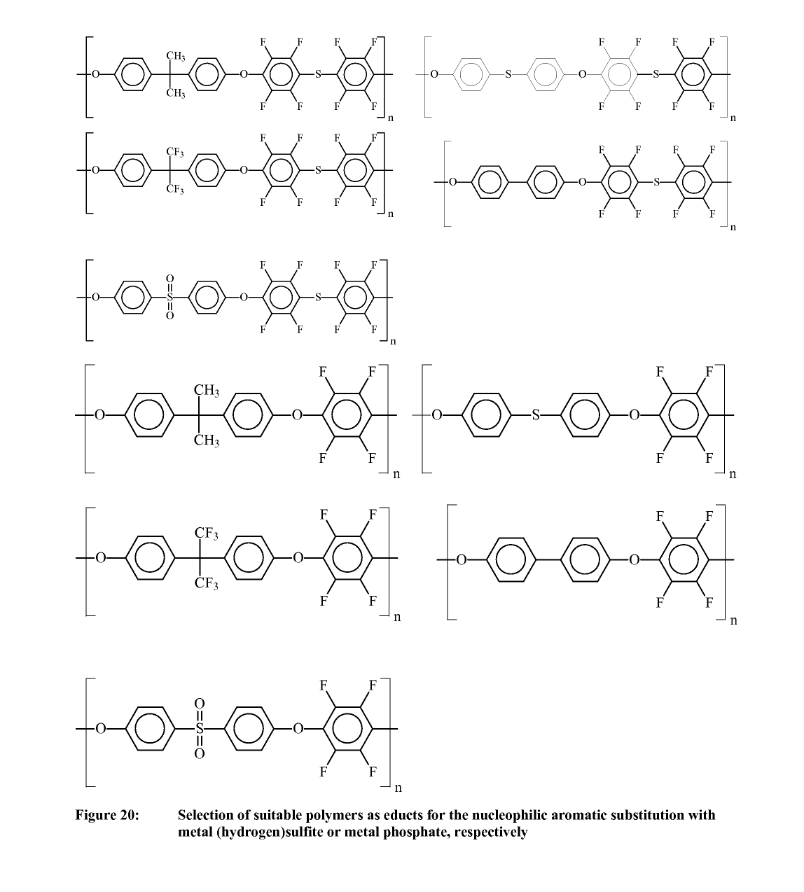

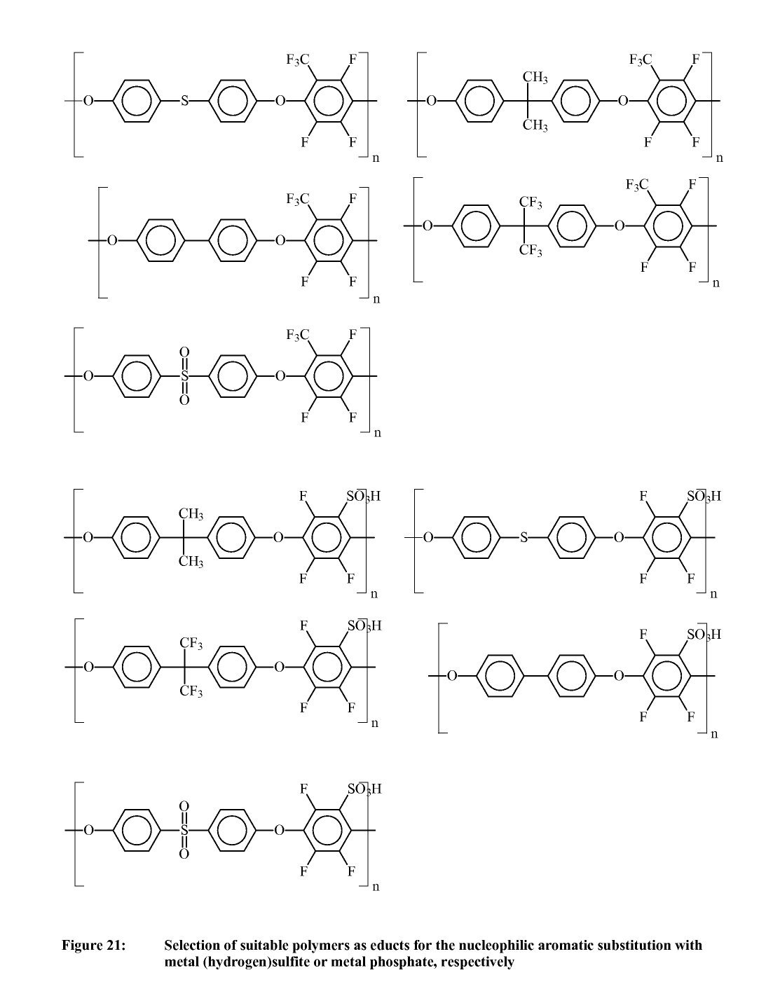

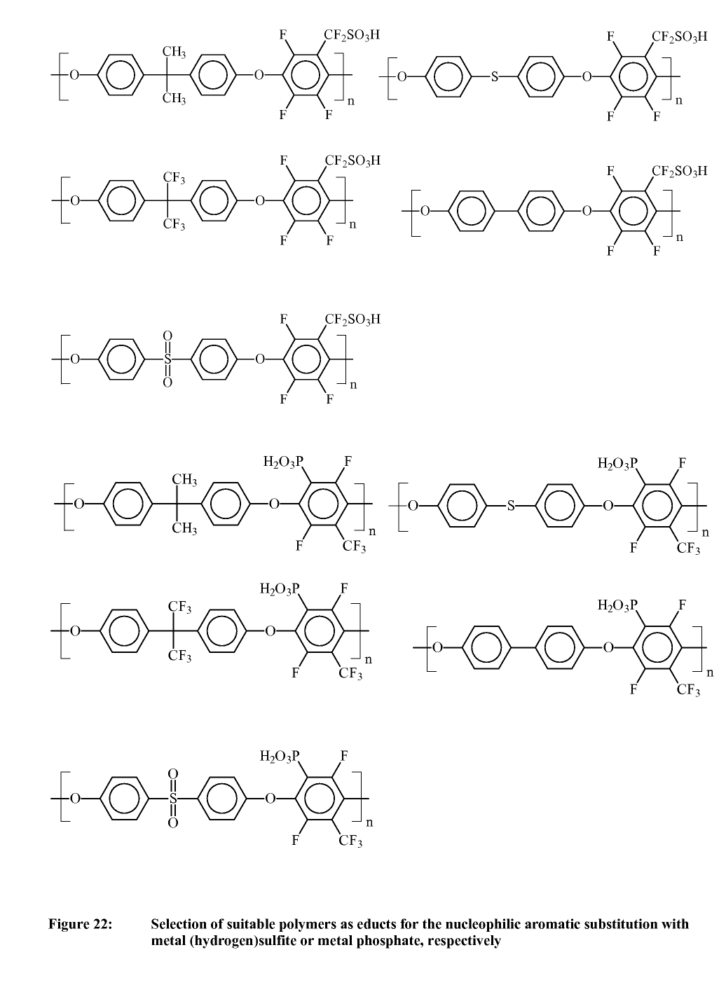

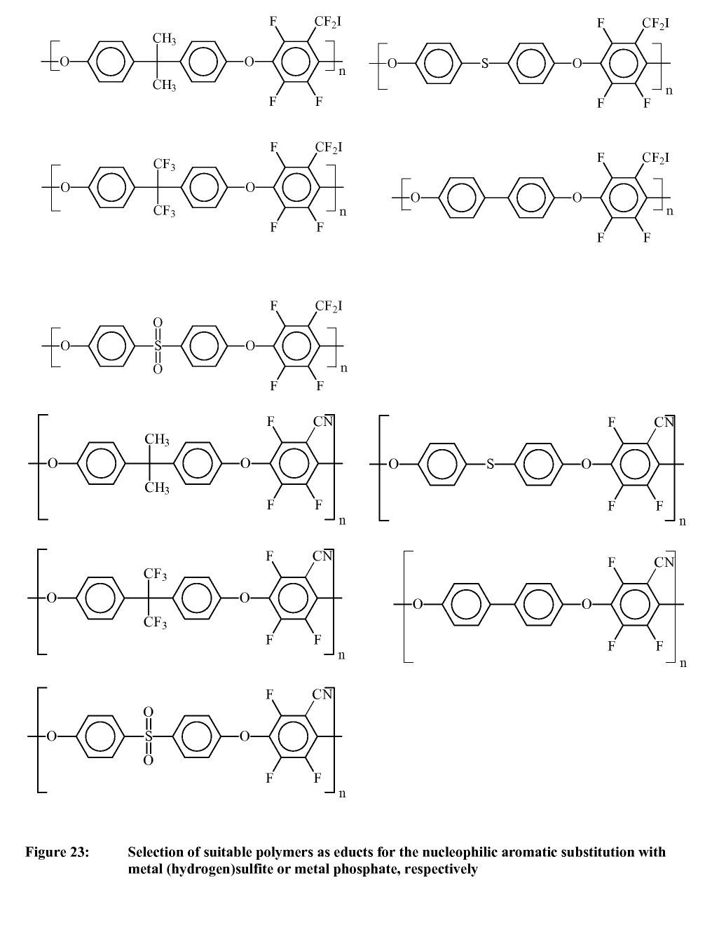

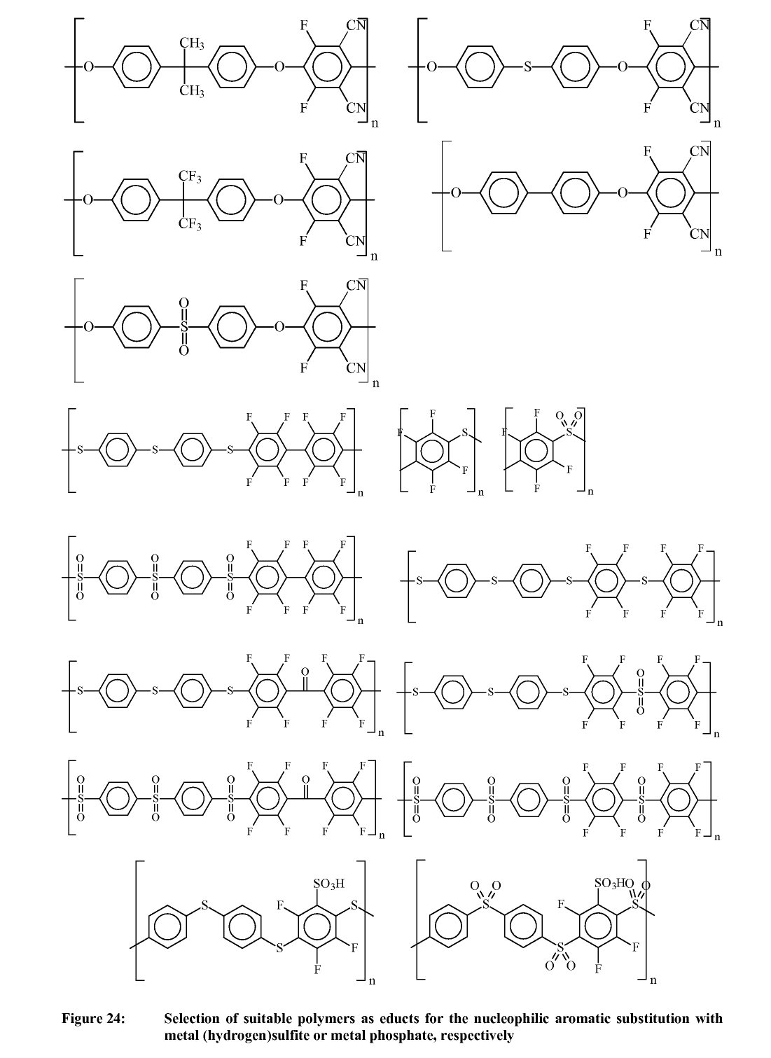

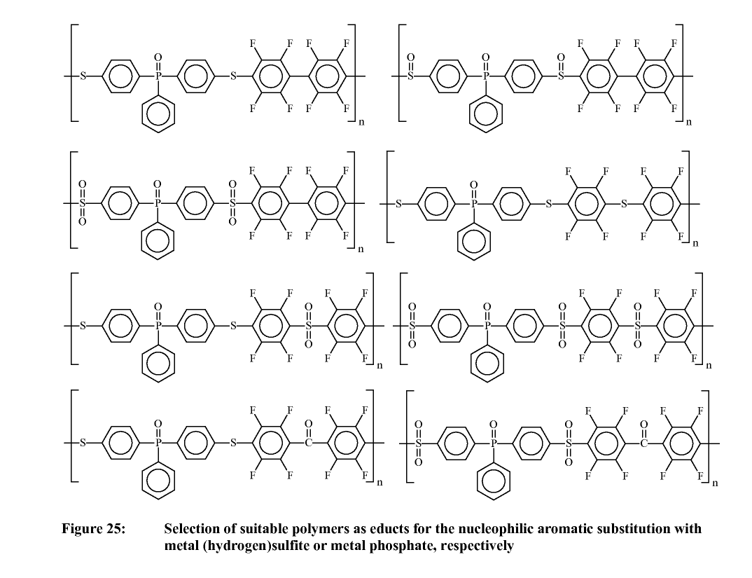

The preferred partly halogenated, especially partly fluorinated aryl polymers for the nucleophilic substitution reaction with metal phosphites, metal sulfites or other metal-sulphur compounds like e.g. sodiumdithionite according to the invention are presented in the following figures (FIG. 6, FIG. 7, FIG. 8, FIG. 9, FIG. 10, FIG. 11, FIG. 12, FIG. 13, FIG. 14, FIG. 15, FIG. 16, FIG. 17, FIG. 18, FIG. 19, FIG. 20, FIG. 21, FIG. 22, FIG. 23, FIG. 24, FIG. 25).

Also statistical copolymers and blockcopolymers, containing the repeating units from figures FIG. 6, FIG. 7, FIG. 8, FIG. 9, FIG. 10, FIG. 11, FIG. 12, FIG. 13, FIG. 14, FIG. 15, FIG. 16, FIG. 17, FIG. 18, FIG. 19, FIG. 20, FIG. 21, FIG. 22, FIG. 23, FIG. 24, FIG. 25, are suitable for the nucleophilic substitution reaction with metal phosphites, metal sulfites or other metal-sulphur compounds like e.g. sodiumdithionite according to the invention as has been found surprisingly.

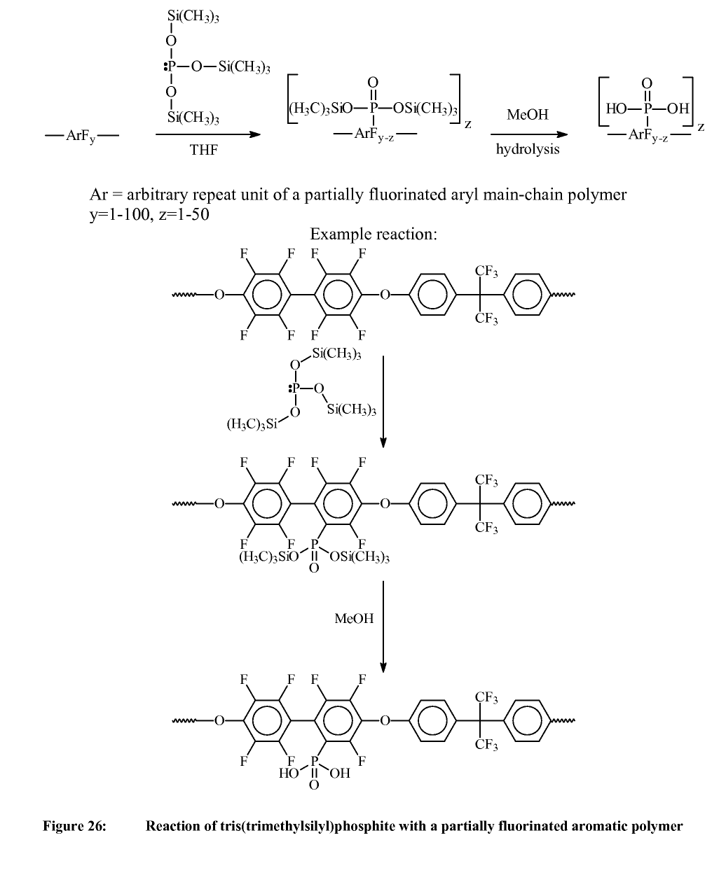

It was furthermore surprisingly, that also non salt-like phosphite compounds are suitable for the nucleophilic aromatic substitution reaction. Thus the compound tris(trimethylsilyl)phosphite and any other silylphosphites can nucleophilicly substitute aromatically bound halogene atoms with a phosphonic acid group (FIG. 26).

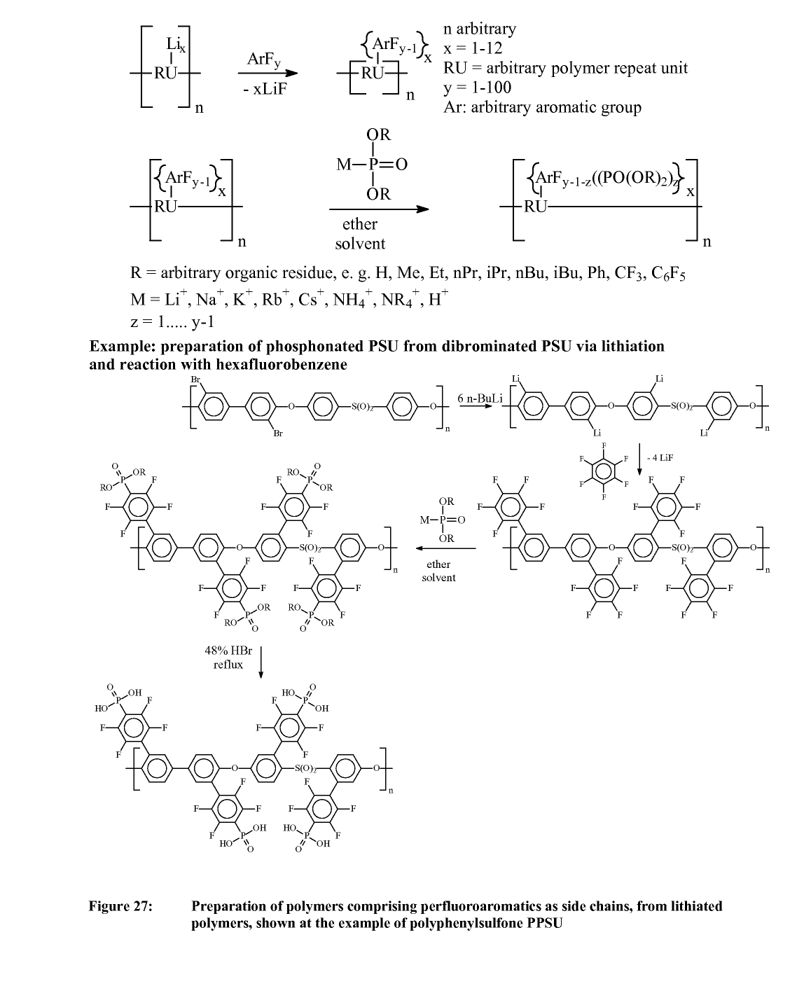

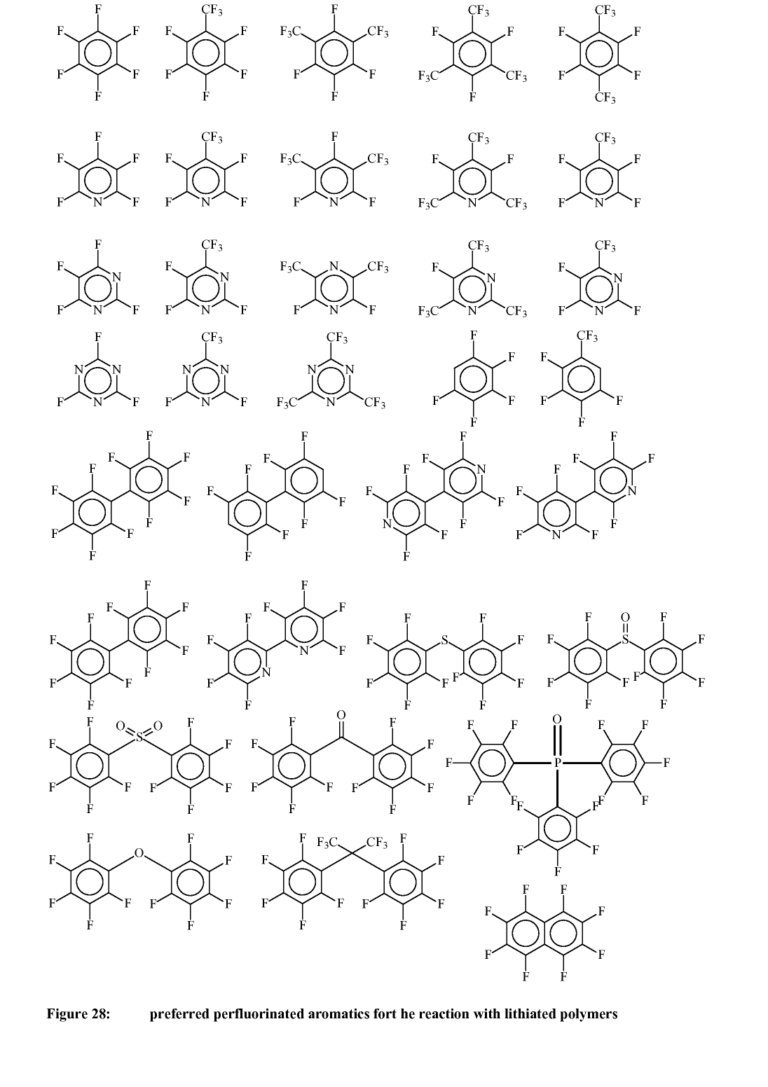

It has been found surprisingly that also polymers modified with perfluorinated side chains, especially aryl main chain polymers, are suitable for the nucleophilic substitution reaction with metal phosphites, metal sulfites or other metal-sulphur compounds like e.g. sodiumdithionite according to the invention. Thereby these polymers with perfluorinated aromates in the side chain can be produced surprisingly e.g. by reaction of the corresponding lithiated polymer with perfluorinated aromates (schema see FIG. 27). So far in the literature only the reaction of low molecular sulfone-stabilised carbaniones with part- or perfluorinated aromates has been described²². The production of these polymers from lithiated polymers is been shown as an example from brominated PPSU Radel R with the perfluoroaromate Hexafluorobenzene in FIG. 27. The preferred part- or perfluorinated aromates fort the reaction with a lithiated polymer are shown in FIG. 28.

### APPLICATION EXAMPLES

### 1. Sulfonated Polysulfone from Sulfinated Polysulfone, Decafluorobiphenyl and Sodium Sulfite

Polysulfon is metalated via the state of the art and reacted with SO2 yielding a polymeric sulfinic acid. The sulfinate group is in ortho position to the sulfone group of polysulfone. The polymeric Li salt is filtered off and is dried under vacuum at low temperature. For the further reaction a polysulfone with 1.5 Li sulfinate groups per polymer repeat unit (RU) is used.

10 g decafluorobiphenyl are mixed with 50 g NMP at room temperature. 10 g of the polymeric sulfinate comprising 1.5 sulfinate groups per RU are dissolved in 90 g NMP. The mixture of decafluorobiphenyl and NMP is vigorously stirred (stirring speed 300 rotations per minute), and the polymeric sulfinate is added slowly (1 ml per minute) added via a dropping funnel. The mixture is stirred further and subsequently heated up to 120° C. (heating rate 1° C. per minute). This heating temperature is kept for 10 hours. Subsequently the mixture is cooled down to 10° C., and 500 ml of a cold-saturated aqueous sodium sulfite solution is added. Subsequently the mixture is heated to 110° C. and kept at this temperature for 10 hours. After cooling down the solvent is evaporated in a rotating evaporator under vacuum. The obtained product is mixed with 1 L of water and dialyzed in a dialysis tube (exclusion limit of the dialysis membrane is 3000 Dalton). Via this procedure the small molecules are separated from the produced sulfonated polymer. After evaporation of the water from the aqueous solution inside the dialysis tube a sulfonated polymer in sodium salt form is obtained.

### 2. Michaelis-Becker Reaction of a Partially Fluorinated Aryl Main-Chain Polymer with Sodium Phosphite (Synthesis of XFS001A)

### Properties and Used Quantities of Educts/Products:

| | | Dimethyl | | | |
|---|---|---|---|---|---|
| | PFS001B | phosphite | Sodium hydride | XFS001A | |
| MW [g mol⁻¹] | 630.34 | 110.05 | 23.99 | 810.42/ru | |
| density [g ml⁻¹] | N/A | 1.200 | N/A | N/A | |
| at 25° C. | | | | | |
| Molar | 10.815 | 21.63 | 21.63 | theoretical: | experimental: |
| proportion | | | | 10.815 | |
| [mmol] | | | | | |
| Mass [g] | 6.8180 g | 2.3807 g | 0.5190 g | 8.7647 g | |
| Volume [ml] | | ≈2.0 ml | | | |

2 equivalents NaPO(OMe)₂ referred to PFS001B

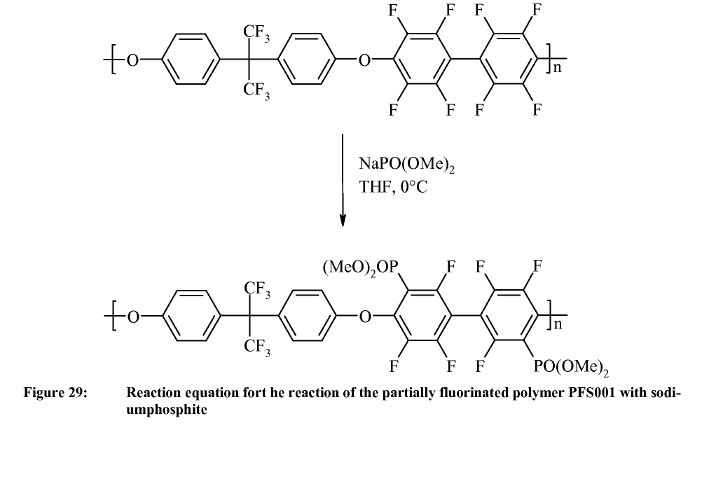

The reaction equation of this reaction is shown in FIG. 29.

To a solution of 0.519 g (21.63 mmol) sodium hydride in 40 ml anhydrous THF are slowly added 21.3807 g (21.63 mmol) Dimethylphosphite (in 40 ml anhydrous THF) via dropping funnel under protective gas atmosphere at 0° C.

After the H₂ formation is finished, the reaction mixture is warmed up to ambient temperature, and via 20 min a solution of 6.8180 g (10.815 mmol) PFS001B in 80 ml anhydrous THF is dropped to the reaction mixture (PFS001B in THF: yellowish, during dropping into the sodium dimethylphosphite solution the reaction mixture becomes pink/orange).

The reaction mixture is stirred overnight at room temperature and is warmed for 3 further hours up to 65° C. (after that, the solution showed a yellow colour, moreover a finely dispersed solid was present (sodium fluoride?) which could however not be filtered off.

Subsequently the solution is concentrated at the rotation evaporator. It was attempted to take up the residuals in roughly 300 ml methylene chloride (to shake with water and to remove the formed NaF). However, a yellowish precipitate (not typical for salt) remained undissolved. The precipitate was filtered off, suspended in 200 ml water and dialysed (XFS001A, CH₂Cl₂-insoluble fraction XFS001A-UF). The CH₂Cl₂-filtrat was concentrated once more, the residual also re-suspended in roughly 200 ml water, and dialysed (XFS001A, CH₂Cl₂-soluble fraction=XFS001A-LF).

### Characterisation

### A. Elemental Analysis (XFS001A-UF):

Sum formula: C₃₁H₂₀O₈F₁₂P₂ (2 Phosphonic acid groups, M=810.14 g mol⁻¹)
Sum formula: C₂₉H₁₄O₅F₁₃P (1 Phosphonic acid group, M=720.37 g mol⁻¹)

| | Theoretical | Theoretical content | Experimental | |
|---|---|---|---|---|
| | content [%] for | [%] for | content | |
| XFS001A-UF | 1 group | 2 groups | [%] | |
| C | 48.35 | 45.94 | 47.26 | 47.29 |
| H | 1.96 | 2.49 | 2.15 | 2.12 |
| O | 11.10 | 15.79 | N/A | N/A |
| F | 34.28 | 28.13 | N/A | N/A |
| P | 4.30 | 7.64 | 3.81 | 3.61 |

### B. Elemental Analysis (XFS001A-LF):

Sum formula: C₃₁H₂₀O₈F₁₂P₂ (2 Phosphonic acid groups, M=810.14 g mol⁻¹)
Sum formula: C₂₉H₁₄O₅F₁₃P (1 Phosphonic acid group, M=720.37 g mol⁻¹)

| | Theoretical | Theoretical | Experimental | |
|---|---|---|---|---|
| | content [%] for | content [%] for | content | |
| XFS001A-LF | 1 group | 2 groups | [%] | |
| C | 48.35 | 45.94 | 50.79 | 50.64 |
| H | 1.96 | 2.49 | 2.26 | 2.29 |
| O | 11.10 | 15.79 | N/A | N/A |
| F | 34.28 | 28.13 | N/A | N/A |
| P | 4.30 | 7.64 | 2.00 | 1.99 |

### 3. Michaelis-Becker Reaction of a Partially Fluorinated Aryl Main-Chain Polymer with Sodium Phosphite (Synthesis of XFS001D)

### Properties and Used Quantities of Educts/Products:

| | | Diethyl | | |
|---|---|---|---|---|
| | PFS001D | phosphite | Sodium hydride | XFS001D |
| MW [g mol⁻¹] | 630.34 | 138.10 | 23.99 | 866.52/ru (2 PO(OEt)₂ per ru) |

| | | | | | |
|---|---|---|---|---|---|
| Molar | 10.815 | 48.15 | 48.15 | theoretical: | experimental: |
| proportion | | (43.26) | (43.26) | 10.815 | 9.404 (87%) |
| [mmol] | | | | | |
| Mass [g] | 6.8180 g | 6.6489 g | 1.155 g | 9.3714 g | 8.149 g |
| Volume [ml] | | (5.9742 g) | (1.0378 g) | | |

Values in brackets: calculated amount, values above: actually used quantities
Remark: The used THF (Fisher) obviously had a higher H₂O-content, since at addition of THF to the weighed NaH a more or less vigorous H2 development begins. Therefore the amount of the polymer PFS001D was not calculated onto the effectively weighed amount (to ensure that actually a 4 fold excess of NaPO(OEt)₂ exists).

4 eq NaPO(OEt)₂ referred to PFS001D

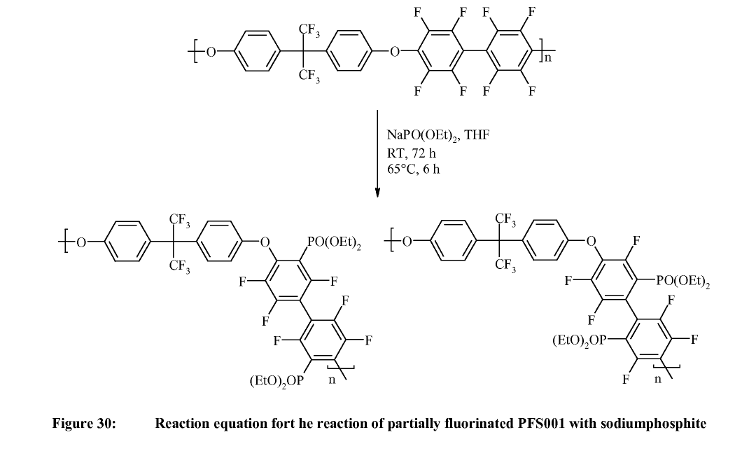

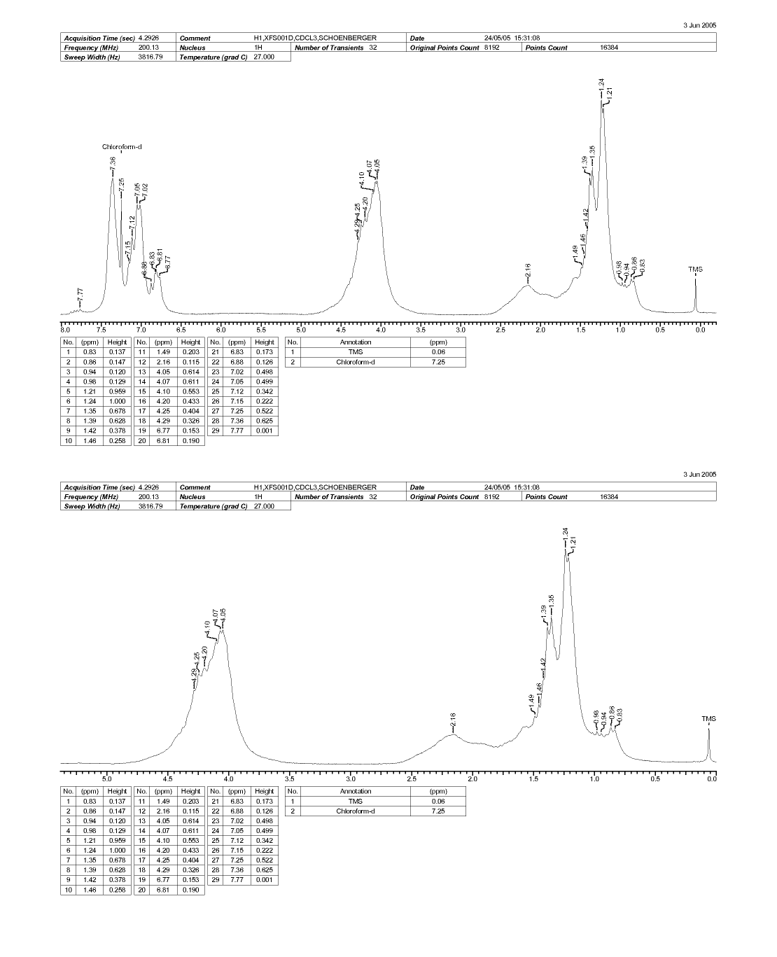

The reaction equation of this reaction is shown in FIG. 30.

To a solution of 1,155 g (48.15 mmol) sodium hydride in 80 ml anhydrous THF are slowly added 6.6489 g (48.15 mmol) Diethylphosphite (in 80 ml anhydrous THF) via dropping funnel under protective gas atmosphere at 0° C.

After the H₂ formation is finished, the reaction mixture is warmed up to ambient temperature, and via 20 min a solution of 6.8180 g (10.815 mmol) PFS001B in 80 ml anhydrous THF is dropped to the reaction mixture (PFS001B in THF: yellowish, during dropping into the sodium dimethylphosphite solution the reaction mixture becomes pink/orange).

The reaction mixture is stirred 72 h at room temperature and is warmed for 6 further hours up to 65° C. Subsequently the solution is concentrated at the rotation evaporator.

The precipitate was suspended in 300 ml water and dialyzed. After evaporation of the solvent the polymer is dried at 60° C. in the vacuum oven overnight.

### Characterisation

### A. Elemental Analysis

Sum formula: C₃₁H₁₈O₅F₁₃P (1 PO(OEt)₂-Group per RU, M=748.42 g mol⁻¹)
Sum formula: C₃₅H₂₈O₈F₁₂P₂ (2 PO(OEt)₂-Groups per RU, M=866.52 g mol⁻¹)
Sum formula: C₃₉H₃₈O₁₁F₁₁P₃ (3 PO(OEt)₂-Groups per RU, M=984.62 g mol⁻¹)
Sum formula: C₄₃H₄₈O₁₄F₁₀P₄ (4 PO(OEt)₂-Groups per RU, M=1102.71 g mol⁻¹)

| | Theoretical | Theoretical | Theoretical | Theoretical |
|---|---|---|---|---|
| | content [%] | content [%] | content [%] for | content [%] for |
| XFS001D | for 1 group | for 2 groups | 3 groups | 4 groups |
| C | 49.75 | 48.51 | 47.57 | 46.84 |
| H | 2.42 | 3.26 | 3.89 | 4.39 |
| O | 10.69 | 14.77 | 17.87 | 20.31 |
| F | 33.00 | 26.31 | 21.22 | 17.23 |
| P | 4.14 | 7.15 | 9.44 | 11.24 |

| | |
|---|---|
| | Experimental |
| | content |
| XFS001D | [%] |

| | | |
|---|---|---|
| C | 48.58 | 48.76 |
| H | 3.71 | 3.78 |
| O | N/A | N/A |
| F | N/A | N/A |
| P | 6.79 | 6.82 |

### B. ¹H-NMR

### See FIG. 30

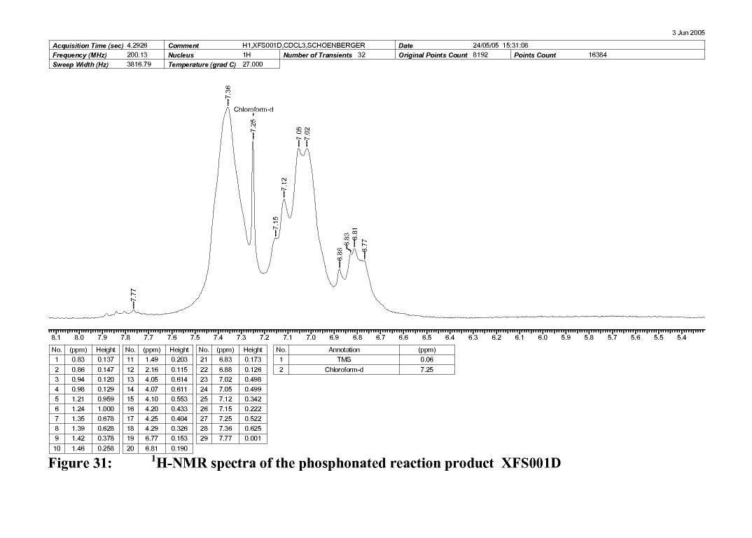

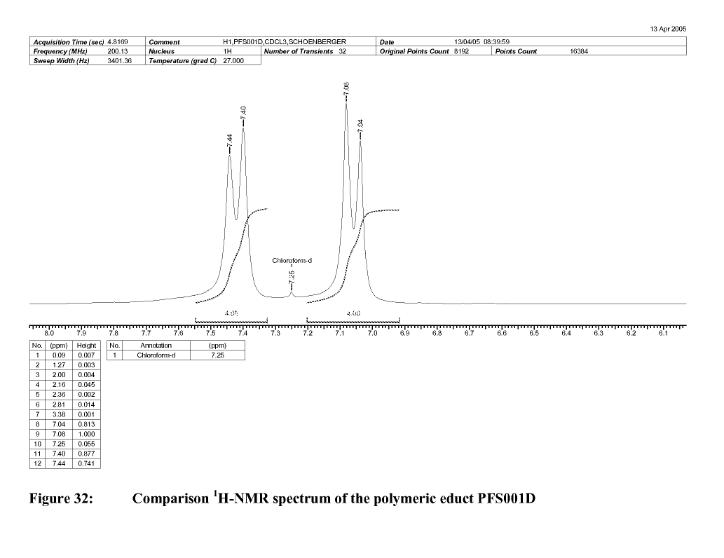

Comparison spectrum PFS001D see FIG. 32

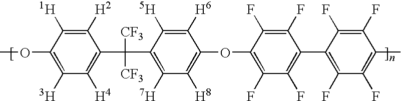

| | |
|---|---|
| solvent: | CDCl₃ |
| reference: | TMS |
| δ [ppm] (200.13 MHz): | 7.06 (d, J = 8.51 Hz, ¹H, ³H, ⁶H, ⁸H, 4 H) |
| | 7.42 (d, J = 8.72 Hz, ²H, ⁴H, ⁵H, ⁷H, 4 H) |

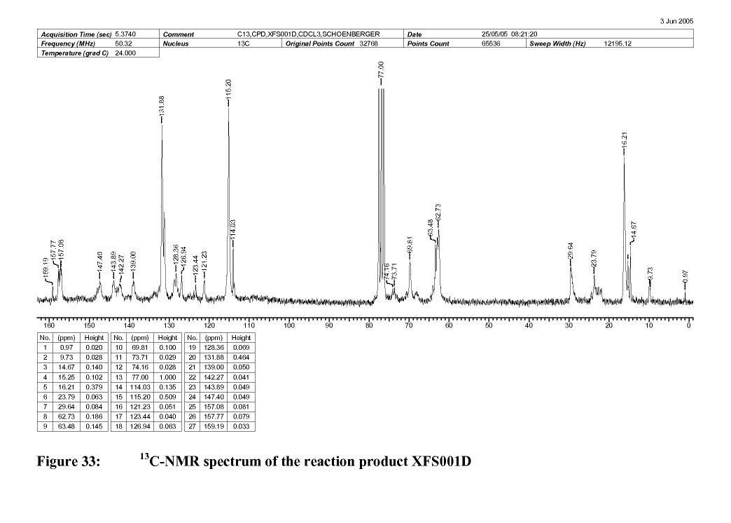

### C. ¹³C-NMR See FIG. 33

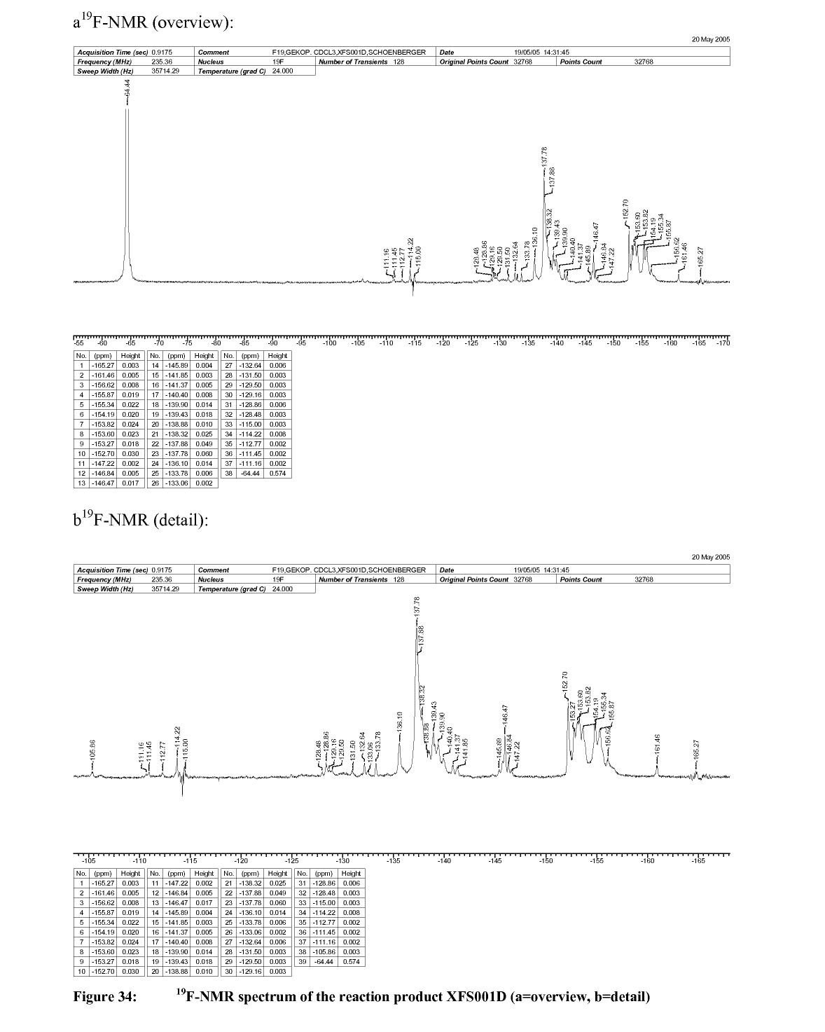

### D. ¹⁹F-NMR See FIG. 34

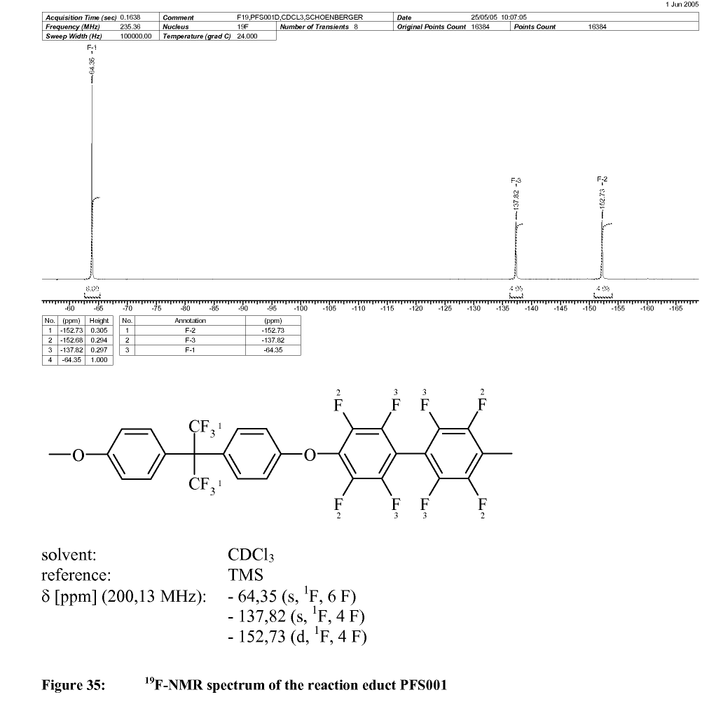

¹⁹F-NMR comparison spectrum PFS001D see FIG. 35

### F. IR Spectra

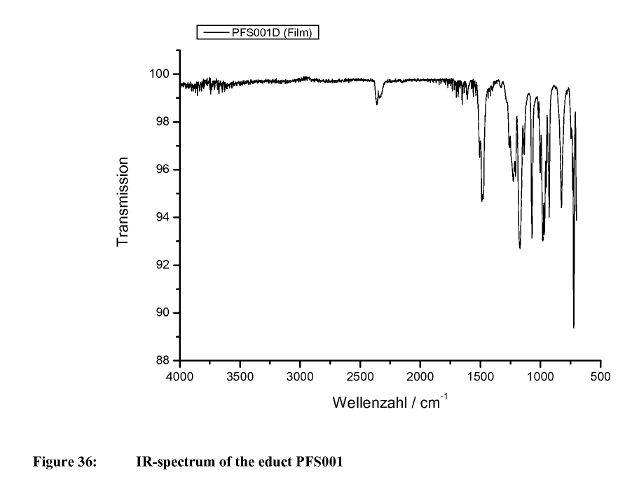

The FTIR spectrum of the educt PFS001 is found in FIG. 36.

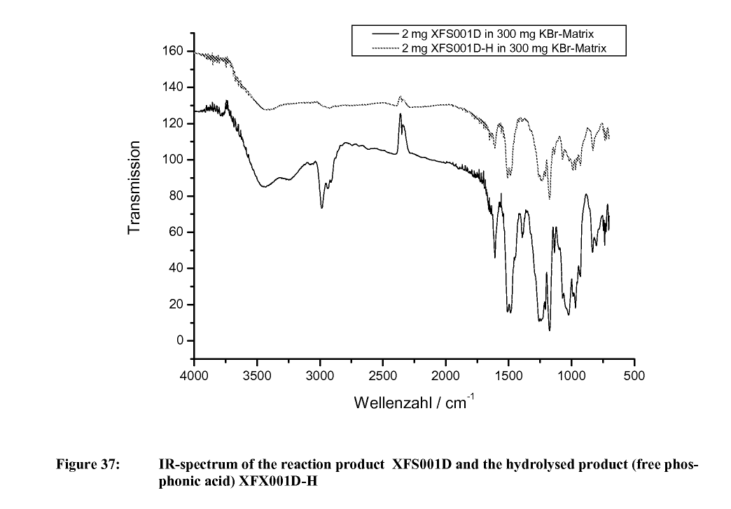

In FIG. 37 the IR spectra of the reaction product XFS001D and the hydrolysed product (free phosphonic acid) XFX001D-H are shown.

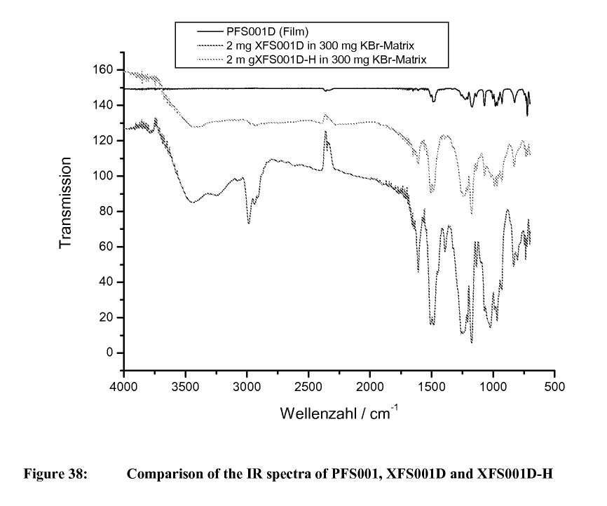

FIG. 38 contains, for comparison, the IR spectra of PFS001, XFS001D and XFS001D-H (free phosphonic acid).

The band at 2983-2912 cm⁻¹ (red curve XFS001D-H) could be the O—H-stretching vibration of the phosphonic acid group. A likewise new appearing peak at 1394 cm⁻¹ cannot be assigned for sure. Following the literature (Hesse, Meier, Zeeh) the P═O-stretching vibration of the phosphonic acid should lie at 1240-1180 cm⁻¹. In the ester form (green curve XFS001D) such a vibration should also be visible (possibly a little bit shifted, because the substitution patterns are different, the peak should however then be shifted relatively far!).

### Hydrolysis of XFS001D (XFS001D-H):

3.50 g XFS001D are suspended in 80 ml 48% hydrobromic acid HBr and heated for 16 h to 100° C. The reaction solution is diluted with 800 ml water, and the precipitate is filtered. The precipitate is resuspended in water and dialysed for 5 days. Subsequently the polymer is dried in the air circulation oven at 80° C. (drying temperature <110° C. to avoid condensation of the phosphonic acids).

Yield (after Dialysis):

2,507 g

### Characterisation of XFS001D-H:

### A. Elemental Analysis

Sum formula: C₂₇H₁₀O₅F₁₃P (1 PO(OH)₂-Group per RU, M=692.32 g mol⁻¹)
Sum formula: C₂₇H₁₂O₈F₁₂P₂ (2 PO(OH)₂-Groups per RU, M=754.31 g mol⁻¹)

| | Theoretical | Theoretical | Experimental | |
|---|---|---|---|---|
| | content [%] for | content [%] for | content | |
| XFS001D-H | 1 group | 2 groups | [%] | |
| C | 46.84 | 42.99 | 43.75 | 43.94 |
| H | 1.46 | 1.60 | 2.76 | 2.81 |
| O | 11.55 | 16.97 | N/A | N/A |
| F | 35.67 | 30.22 | N/A | N/A |
| P | 4.47 | 8.21 | 5.72 | 5.85 |

### B. Measurement of the IEC-Value

| IEC_{direct} [meq g⁻¹] | IECₒᵥₑᵣₐₗₗ [meq g⁻¹] |
|---|---|
| 1.11^{[1]} | 3.59 |

^{[1]}The polymer was buoying upwards during the measurement (exchange complete?)

### 4. Reaction of Octafluorotoluene with Lithiated PSU (AK-51)

### Educts:

22.1 g PSU Udel P 1800 (0.05 mol) dried
800 ml THF anhydrous
10 ml n-BuLi 10 N (0.1 mol)
28.4 ml=47.2 g octafluorotoluene (0.2 mol, MW=236 g/mol)

### Procedure:

Under protective gas the THF is loaded into the reaction flask. Subsequently the dried polymer is introduced into the reaction flask with stirring and vigorous rinsing with argon. After the polymer is dissolved, it is cooled to −50° C. (T as low as possible, with vigorous argon stream). Then the polymer solution is titrated cautiously with 2.5N n-BuLi until a slight yellow/orange coloration shows that the solution is water-free. Then the 10N n-BuLi is introduced with syringe within 10 min. The solution is stirred for 2 hours.

Then the octafluorotoluene is introduced via syringe (solution is getting highly viscous). One awaits how the color of the reaction mixture is changing. If the color does not change, one lets warm up to −30° C. overnight. The solution is stirred until the reaction mixture is decolourized, at the utmost overnight at −30° C. If the solution is not decolourized, the temperature is increased at the next morning up to maximally −10° C. (the solution remains highly viscous).

20 ml methanol are introduced via syringe until decolourization of the reaction mixture. Then it is warmed up to room temperature. The polymer is precipitated in 2 l methanol, filtered off and washed with methanol. The precipitated polymer is again filtered, dried and stirred in 800 ml methanol. Then it is again filtered off, again resuspended in 400 ml methanol, stirred, filtered off and dried in vacuum at 50° C. From the dried polymer a dissolution experiment is made in NMP (soluble, film-forming properties can be detected). The substitution degree of the modified polymer is determined via ¹H/¹³C-NMR and elemental analysis (C, H, S).

Yield: 35.4 g (81.0% of the theoretical yield of 43.73 g)

### Elemental Analysis

### Calculated onto 2 Groups

C₄₁H₂₀F₁₄O₄S

874.64

874.085876

C, 56.30%; H, 2.30%; F, 30.41%; O, 7.32%; S, 3.67%

| | found | calculated |
|---|---|---|
| C | 56.9 | 56.30 |
| H | 2.90 | 2.30 |
| S | 3.98 | 3.67 |
| F | 19.04 | 30.41 |

### Referring to the Fluorine Content 1.25 Groups Per Repeat Unit are Bound!

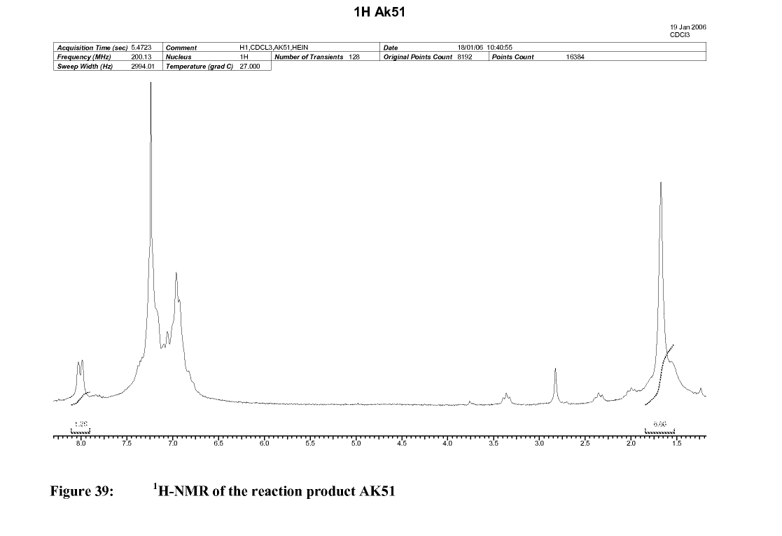

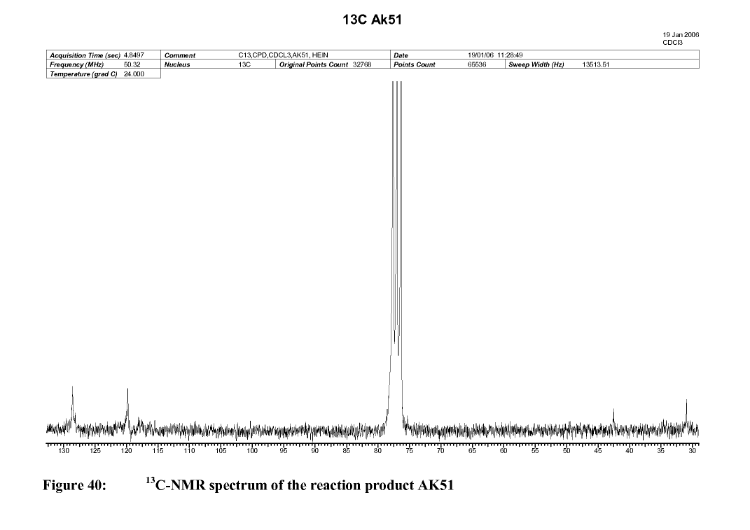

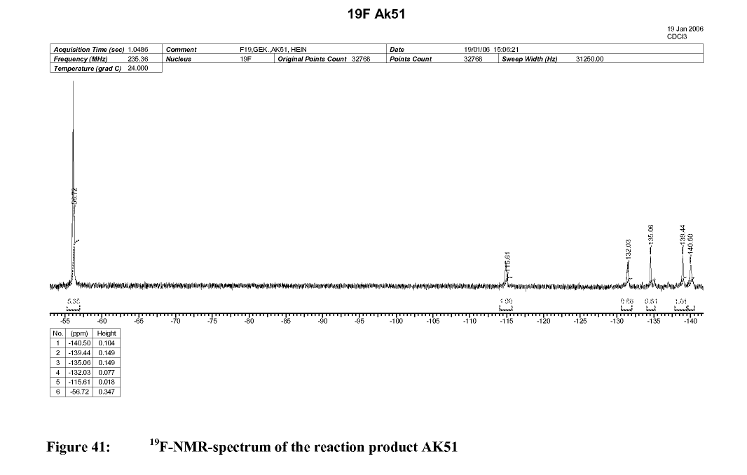

The ¹H-NMR spectrum of the reaction product AK51 is shown in FIG. 39. The ¹³C-NMR spectrum of the reaction product AK51 is found in FIG. 40. The ¹⁹F-NMR-spectrum of the reaction product AK51 is shown in FIG. 41.

### 5. Reaction of Hexafluorobenzene with Lithiated PSU A 1179

### Educts:

11.05 g PSU Udel P 1800 (0.025 mol) dried
800 ml THF anhydrous
5 ml n-BuLi 10 N (0.05 mol)
11.54 ml=18.6 g hexafluorobenzene (0.1 mol, MW=186.056 g/mol)

### Procedure:

Under protective gas the THF is loaded into the reaction flask. Subsequently the dried polymer is introduced into the reaction flask with stirring and vigorous rinsing with argon. After the polymer is dissolved, it is cooled to −50° C. (T as low as possible, with vigorous argon stream). Then the polymer solution is titrated cautiously with 2.5N n-BuLi until a slight yellow/orange coloration shows that the solution is water-free. Then the 10N n-BuLi is introduced with syringe within 10 min. The solution is stirred for 2 hours.

Then the hexafluorobenzene is introduced via syringe (solution is getting highly viscous). One awaits how the color of the reaction mixture is changing. If the color does not change, one lets warm up to −30° C. overnight. The solution is stirred until the reaction mixture is decolourized, at the utmost overnight at −30° C. (decrease ¹⁹F-NMR A 1179a: insoluble in CHCl₃, sparingly soluble in DMSO).

If the solution is not decolourized, the temperature is increased at the next morning up to maximally −10° C. (decrease ¹⁹F-NMR A 1179a: insoluble in CHCl₃, medium solubility in DMSO). 20 ml methanol is introduced via syringe until decolourization of the reaction mixture. Then it is warmed up to room temperature.

The polymer is precipitated in 2 l methanol, filtered off, resuspended in 0.5 l MeOH, filtered of, and washed with methanol onto the frit.

The precipitated polymer is dried in vacuum at 50° C. From the dried polymer a dissolution experiment is made in NMP. The substitution degree of the dried polymer is determined via ¹H/¹³C/¹⁹F-NMR and elemental analysis (C, H, S, F).

### Polymer has a Bad Solubility in NMP, Requires 12 h for Complete Dissolution!

Yield: 16.8 g (86.7% of the theoretical yield of 19.37 g)

### Elemental Analysis 1179a (−30° C.)

### Calculated onto 2 Groups

C₃₉H₂₀F¹⁰O₄S

774.62

774.092263

C, 60.47%; H, 2.60%; F, 24.53%; O, 8.26%; S, 4.14%

| | found | calculated |
|---|---|---|
| C | / | 60.47 |
| H | / | 2.60 |
| S | / | 4.14 |
| F | / | 24.53 |

### Elemental Analysis 1179b (−10° C.)

### Calculated onto 2 Groups

C₃₉H₂₀F₁₀O₄S

774.62

774.092263

C, 60.47%; H, 2.60%; F, 24.53%; O, 8.26%; S, 4.14%

| | found | calculated |
|---|---|---|
| C | 63.17 | 60.47 |
| H | 3.98 | 2.60 |
| S | 4.81 | 4.14 |
| F | 19.55 | 24.53 |

### Referring to the Fluorine Content 1.59 Groups Per Repeat Unit are Bound!

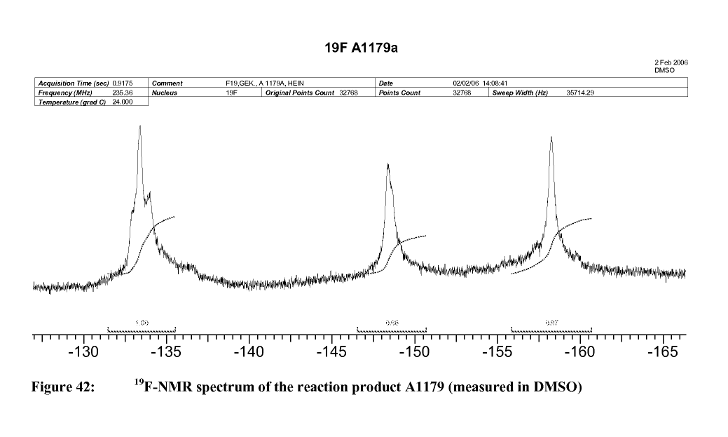

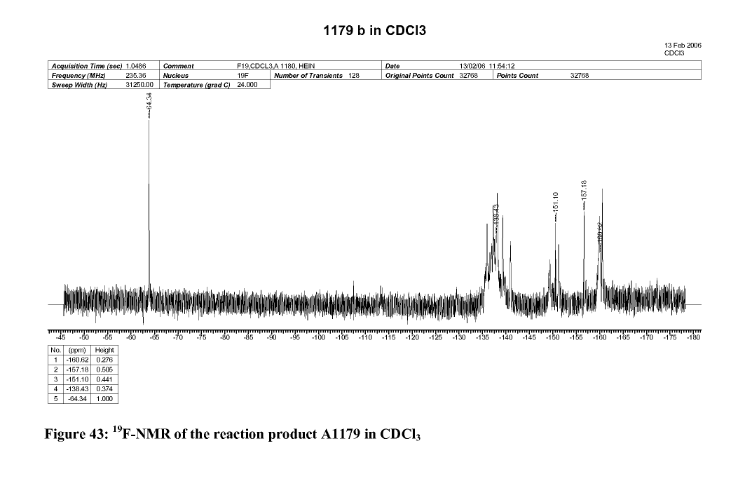

The ¹⁹F-NMR spectrum of the reaction product A1179 in the solvent shown in FIG. 42. One sees very nicely that the hexafluorobenzene has reacted with the lithiated PSU. One finds three peaks with the approximate integral ratio 2:2:1 (2 ortho-F:2 meta-F:1 para-F). In FIG. 43 the ¹⁹F-NMR spectrum of the reaction product A1179 in the solvent CDCl₃ is shown. One sees that the reaction product is very bad soluble in CDCl₃.

### 6. Reaction of A 1179 (PSU/Hexafluorbenzene/n-BuLi) with Sodiumdiethylphosphite (A1184)

### Educts:

5 g A 1179 with 1.59 groups (M=774.62 g/mol, 6.45 mmol), dissolved/suspended in 100 ml THF
1.78 g Diethylphosphite (M=138.10 g/mol, 12.9 mmol), dissolved in 20 ml THF,
bp=50-51° C. at 2 mm Hg, density: 1,072 g/cm³, refractive index: 1.407
0.31 g sodium hydride (M=24.0 g/mol, 12.9 mmol), dissolved in 20 ml THF

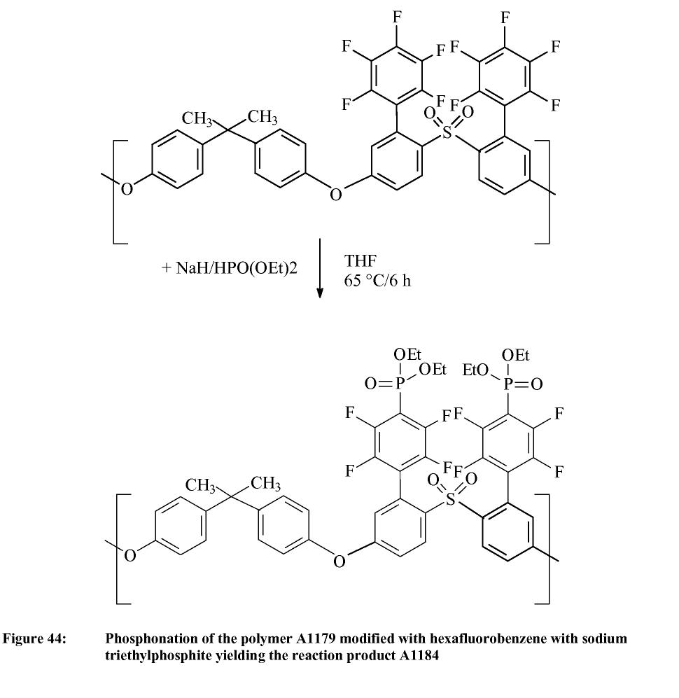

### Reaction Equation: See FIG. 44.

### Procedure

Under protective gas at 0° C. 1.78 g (12.9 mmol) Diethylphosphite, dissolved in 20 ml anhydrous THF, are added into a 250 ml three-neck reaction flask in which are placed 0.31 g (12.9 mmol) NaH, dissolved in 20 ml THF. If no hydrogen develops any more (ca. 30 min), the solution is warmed up to room temperature, and the compound A1179, dissolved in 100 ml THF, is added into the reaction mixture via a dropping funnel. The mixture is then stirred for 6 hours up to 65° C., subsequently hydrolyzed with 20 ml methanol. The THF is removed at the rotary evaporator, and the reaction mixture is resuspended in water, and dialysed for 48 hours (3× water change). The water is evaporated in big porcelain bowls in the oven at 80° C., subsequently in vacuum oven at 80° C.

From the product the following analyses are made: ¹H-, ¹⁹F-, ¹³C-NMR, elemental analysis (C, H, P, F)

| | |
|---|---|
| Yield: | 4.5 g ≈ 6.52 g (69.1% of the theoretical yield) |
| ¹H, ¹³C, ¹⁹F, ³¹P-NMR: | in DMSO A 1184 D medium soluble |
| | in CDCl₃ A 1184 C badly soluble |

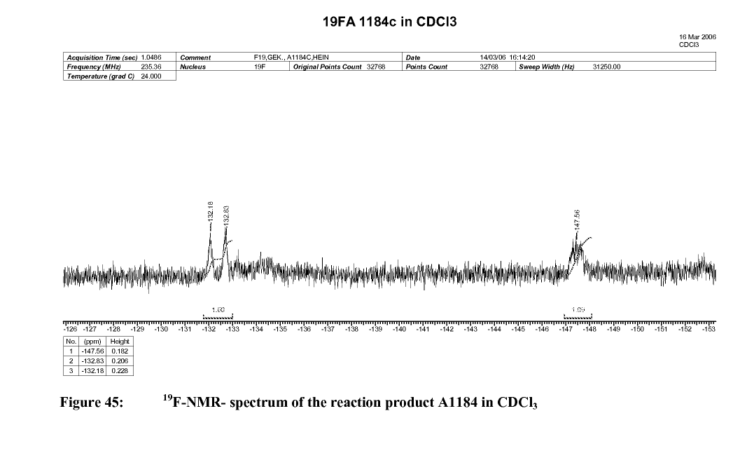

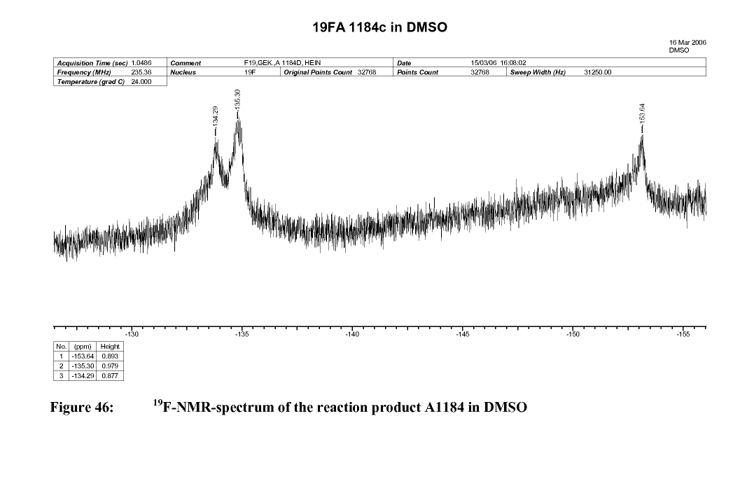

In FIG. 45 the ¹⁹F-NMR-Spectrum of the reaction product A1184 in CDCl₃ is shown. The signals are very weak because of the bad solubility of the reaction product in CDCl₃. In FIG. 46 the ¹⁹F-NMR-Spectrum of the reaction product A1184 in DMSO is shown. In comparison with the ¹⁹F-NMR-Spectrum des reaction educt A1179 in DMSO (FIG. 43) it shows that one signal has disappeared, which indicates the substitution of the, para-F with sodiumdiethylphosphite and therefore the occurrence of the desired substitution reaction.

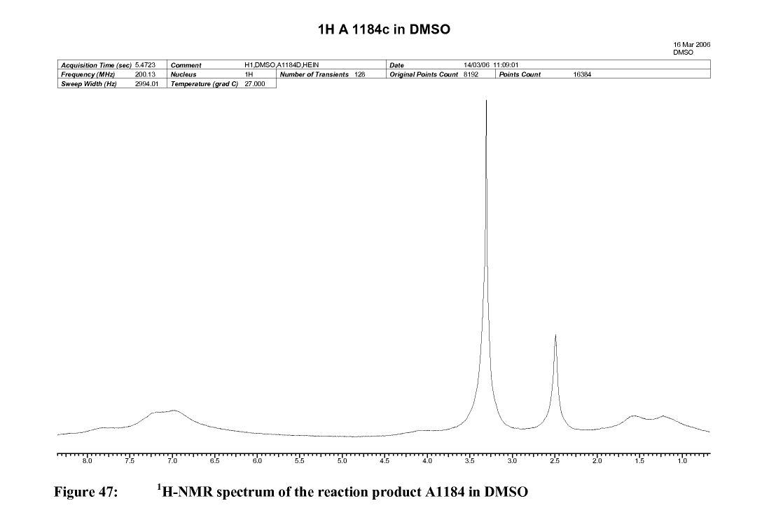

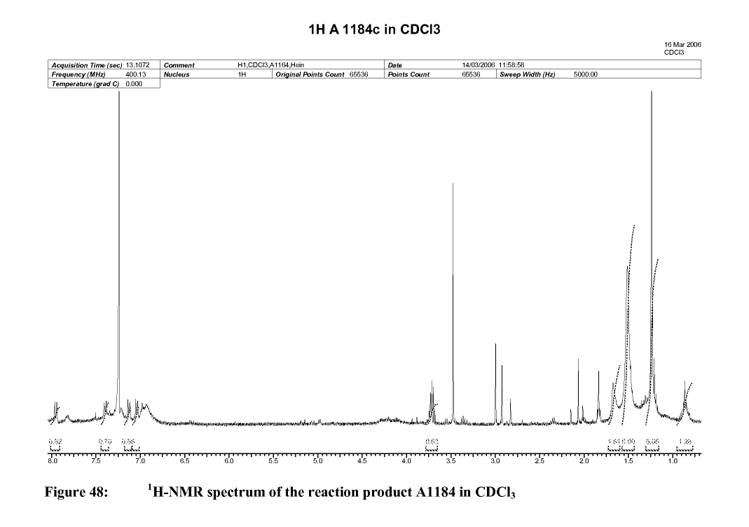

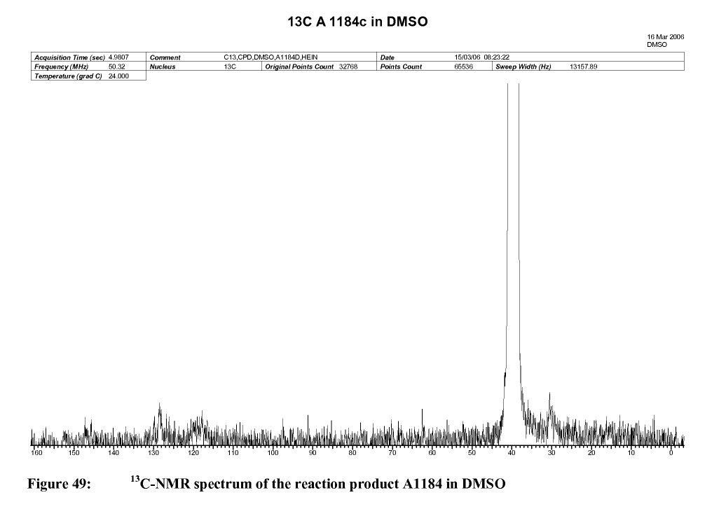

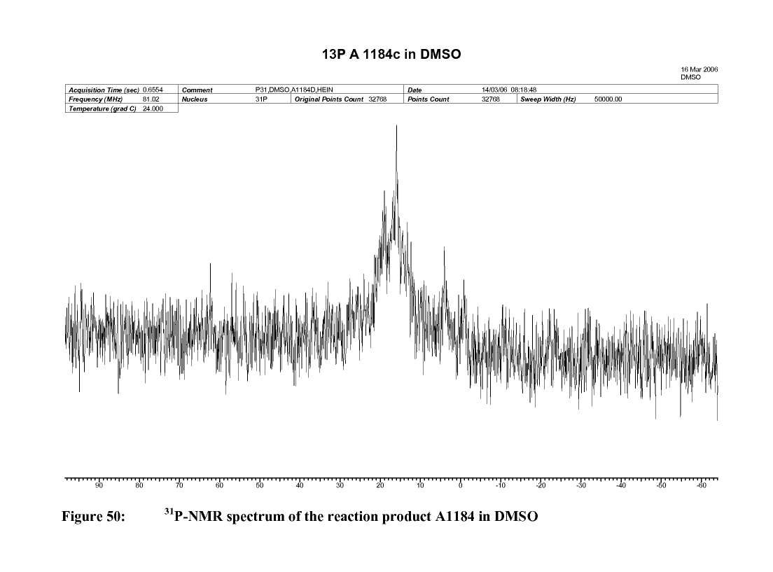

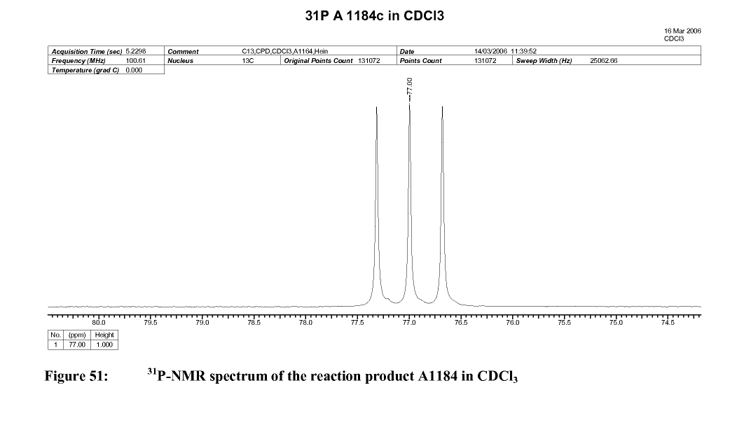

In FIG. 47 the ¹H-NMR spectrum of the reaction product A1184 in DMSO is shown, in FIG. 48 the ¹H-NMR spectrum of the reaction product A1184 in CDCl₃. In FIG. 49 is shown the ¹³C-NMR spectrum of the reaction product A1184 in DMSO, in FIG. 50 the ³¹P-NMR spectrum of the reaction product A1184 in DMSO. In FIG. 51 is shown the ³¹P-NMR spectrum of the reaction product A1184 in CDCl₃. One sees very nicely in FIG. 51 the coupling of the phosphonate-P with the neighboring F.

### Elemental Analysis

### Calculated onto 1 Group

C₃₇H₃₁F₄O₇PS

726.67

726.146426

C, 61.16%; H, 4.30%; F, 10.46%; O, 15.41%; P, 4.26%; S, 4.41%

| | found | calculated |
|---|---|---|
| C | 63.29 | 61.16 |
| H | 4.75 | 4.30 |
| S | 4.34 | 4.41 |
| F | / | 10.46 |
| P | 3.95 | 4.26 |

### Referring to Phosphorus 0.93 Groups Per RU

### Calculated onto 2 Groups

C₄₇H₄₀F₈O₁₀P₂S

1010.82

1010.168972

C, 55.85%; H, 3.99%; F, 15.04%; O, 15.83%; P, 6.13%; S, 3.17%

| | found | calculated |
|---|---|---|
| C | 63.29 | 55.85 |
| H | 4.75 | 3.99 |
| S | 4.34 | 3.17 |
| F | / | 15.04 |
| P | 3.95 | 6.13 |

### Referring to Phosphorus 1.28 Groups Per RU

### 7. Reaction of Decafluorobiphenyl with Lithiated PSU A 1180

### Educts:

5.53 g PSU Udel P 1800 (0.0125 mol) dried
800 ml THF anhydrous
2.5 ml n-BuLi 10 N (0.025 mol)
16.7 g Decafluorobiphenyl (0.1 mol, MW=334.11 g/mol)

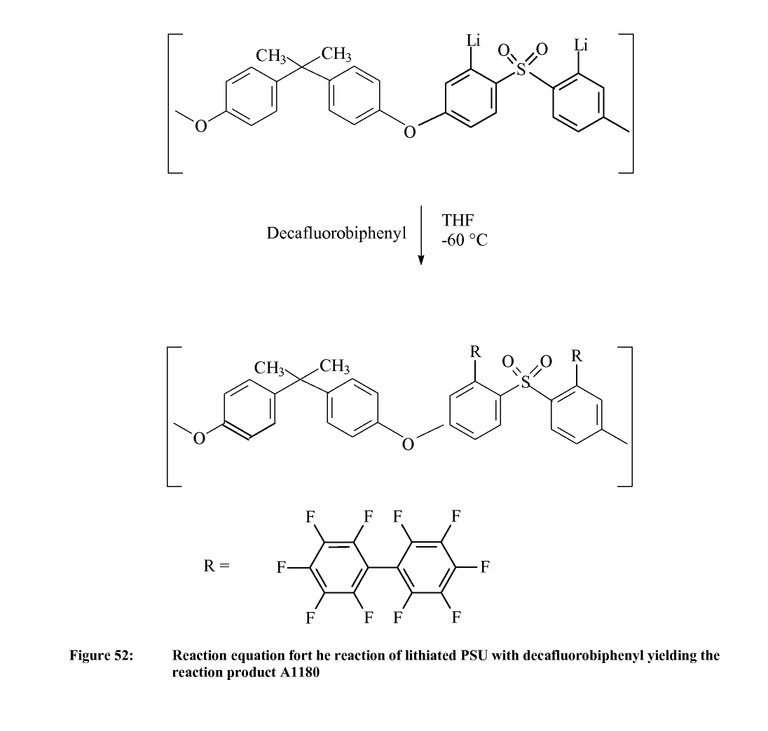

### Reaction Equation: See FIG. 52

### Procedure:

Under protective gas the THF is loaded into the reaction flask. Subsequently the dried polymer is introduced into the reaction flask with stirring and vigorous rinsing with argon. After the polymer is dissolved, it is cooled to −60° C. with vigorous argon stream.

Then the polymer solution is titrated cautiously with 2.5N n-BuLi until a slight yellow/orange coloration shows that the solution is water-free. Then the 10N n-BuLi is introduced with syringe within 10 min. The solution is stirred for 2 hours.

Then the decafluorobiphenyl is introduced via syringe (dissolved in 100 ml THF, dropping funnel), the colour changes spontaneously into black.

After 15 h reaction time at −55° C. the colour has changed/brightened up into light-grey, the reaction is then aborted and hydrolysed.

Therefore 20 ml MeOH are introduced via syringe until the reaction mixture is decolonized. Then the mixture is warmed up to room temperature.

The polymer is precipitated in 2 l meOH, the meOH is rotated off, the mixture is suspended in water and dialysed. Then the water is evaporated at 50° C. and the polymer is dried at 50° C. in vacuum. From the dried polymer a dissolution experiment in NMP is made. The substitution degree of the modified polymer is determined via ¹H/¹³C/¹⁹F-NMR and elemental analysis (C, H, S, F).

Yield: 8.8 g (refers to 63.5% of the theoretical yield of 13.86 g)

| | | |
|---|---|---|
| Solubilities: | insoluble in Acetonitril | |
| | Badly soluble in CHCl₃ | A 1180 (NMR) |
| | gelates in CH₂Cl₂ | |
| | insoluble in D₂O | |
| | insoluble in Aceton | |
| | medium solubility in DMSO | A 1180 D (NMR) |

### Elemental Analysis

### Calculated onto 2 Groups

C₅₁H₂₀F₂₀O₄S

1108.74

1108.076296

C, 55.25%; H, 1.82%; F, 34.27%; O, 5.77%; S, 2.89%

| | found | calculated |
|---|---|---|
| C | 63.63 | 55.25 |
| H | 3.51 | 1.82 |
| S | 4.31 | 2.89 |
| F | 21.30 | 34.27 |

### Referring to the Fluorine Content 1.24 Groups Per Repeat Unit are Bound!

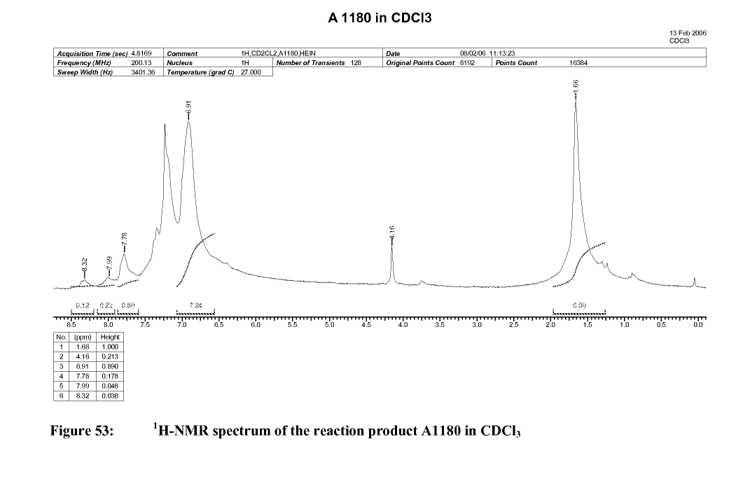

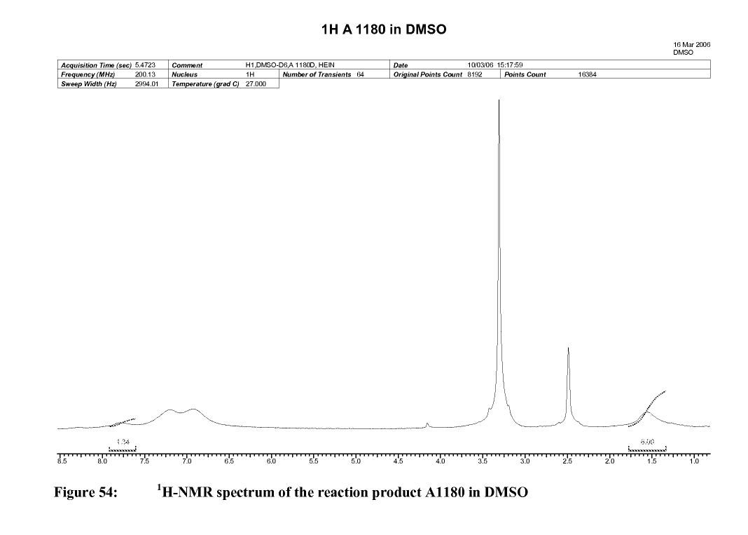

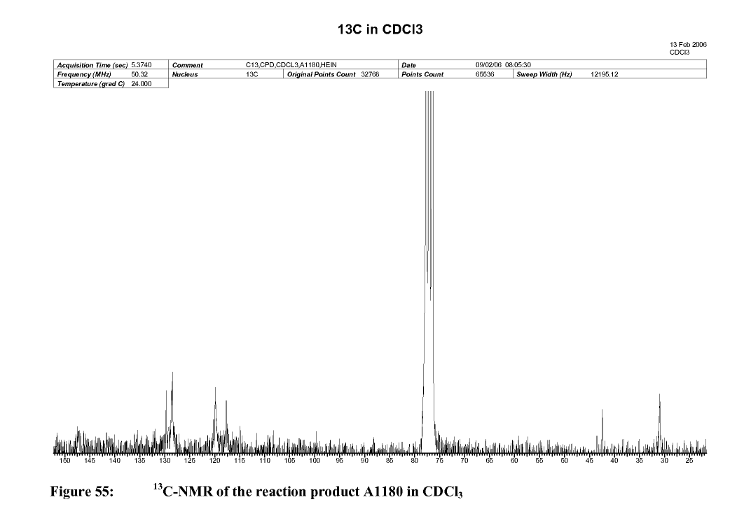

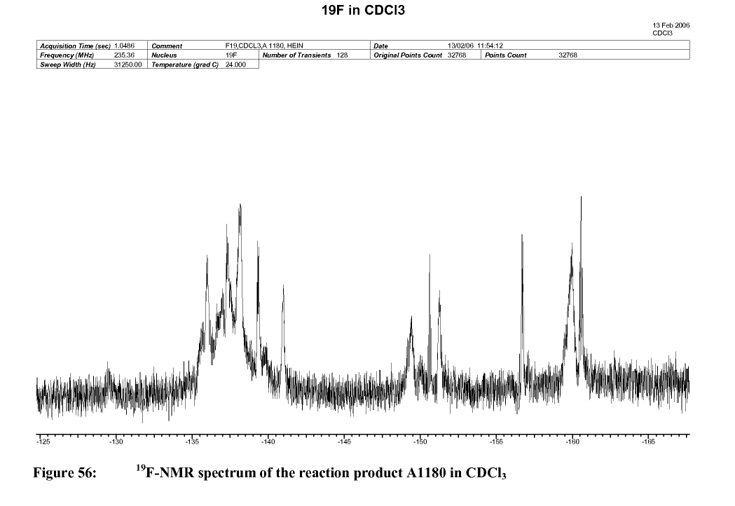

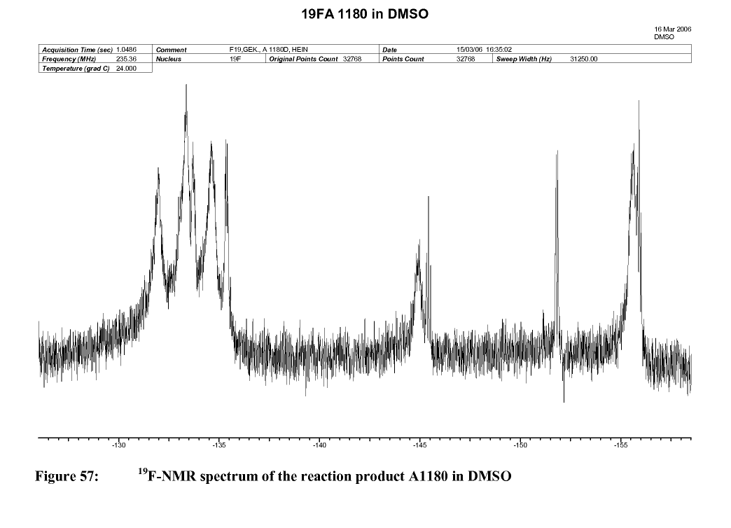

In FIG. 53 the ¹H-NMR spectrum of the reaction product A1180 in CDCl₃ is shown, in FIG. 54 the ¹H-NMR spectrum of the reaction product A1180 in DMSO. In FIG. 55 the ¹³C-NMR spectrum of the reaction product A1180 in CDCl₃ is shown, in FIG. 56 the ¹⁹F-NMR spectrum of the reaction product A1180 in CDCl₃, and in FIG. 57 the ¹⁹F-NMR spectrum of the reaction product A1180 in DMSO.

### 8. Reaction of Pentafluoropyridine with Lithiated PSU A 1181

### Educts:

5.53 g PSU Udel P 1800 (0.0125 mol) dried
800 ml THF anhydrous
2.5 ml n-BuLi 10 N (0.025 mol)
8.45 g=5.3 ml Pentafluoropyridine (0.05 mol, MW=169.05 g/mol)

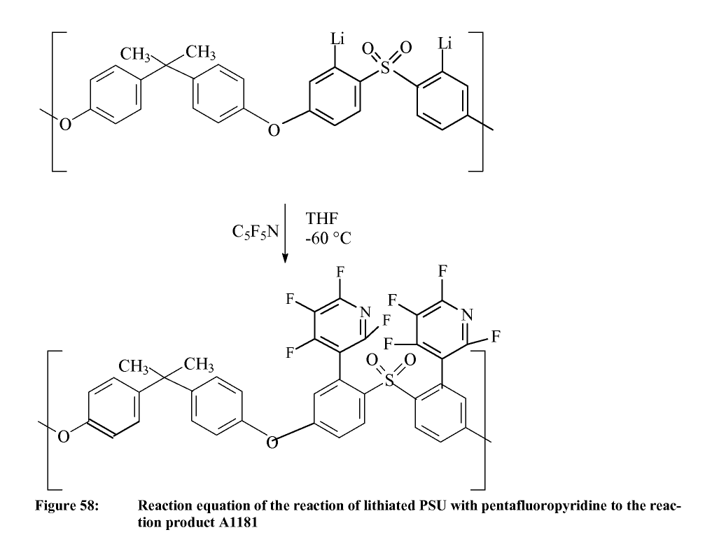

### Reaction Equation: See FIG. 58

### Procedure:

Under protective gas the THF is loaded into the reaction flask. Subsequently the dried polymer is introduced into the reaction flask with stirring and vigorous rinsing with argon. After the polymer is dissolved, it is cooled to −60° C. (T as low as possible, with vigorous argon stream). Then the polymer solution is titrated cautiously with 2.5N n-BuLi until a slight yellow/orange coloration shows that the solution is water-free. Then the 10N n-BuLi is introduced with syringe within 10 min. The solution is stirred for 2 hours.

Then the Pentafluoropyridine is added via a dropping funnel (dissolved in 50 ml THF). One awaits how the colour of the reaction mixture is changing (reaction time: 4 h, temperature: −60° C.). If the colour does not change, the reaction is continued for 96 h at −55° C. The colour changes from dark red/dark orange to light orange.

20 ml MeOH are introduced via syringe until declourization of the reaction mixture. Then it is warmed up to room temperature.

The polymer is precipitated in 2 l meOH, filtered off, digested with 0.5 l meOH, filtered off, and washed with meOH onto the frit.

The precipitated polymer is dried at 60° C. in vacuum. From the dried polymer a dissolution experiment in NMP is made. The substitution degree of the modified PSU is determined via ¹H/¹³C/¹⁹F-NMR and elemental analysis (C, H, S, F).

Yield: 9.1 g (93.5% of the theoretical yield of 9.73 g)

Solubilities: insoluble in Acetonitril
Badly soluble in CHCl₃
gelates in CH₂Cl₂
insoluble in D₂O
insoluble in Acetone
medium solubility in DMSO

| | |
|---|---|
| ¹H, ¹³C-NMR: | A 1181 D in DMSO |
| | A 1181 C in CDCl₃ |

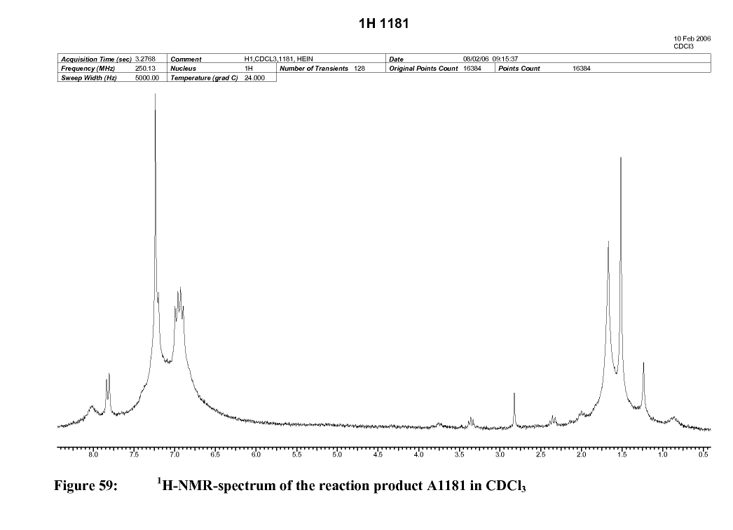

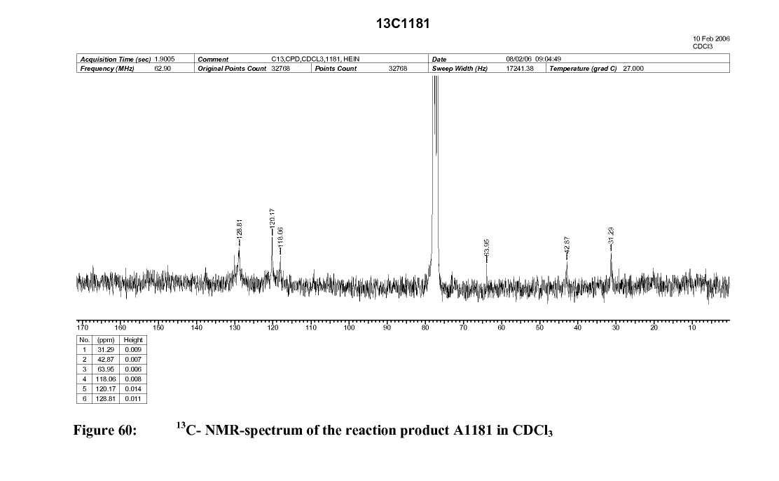

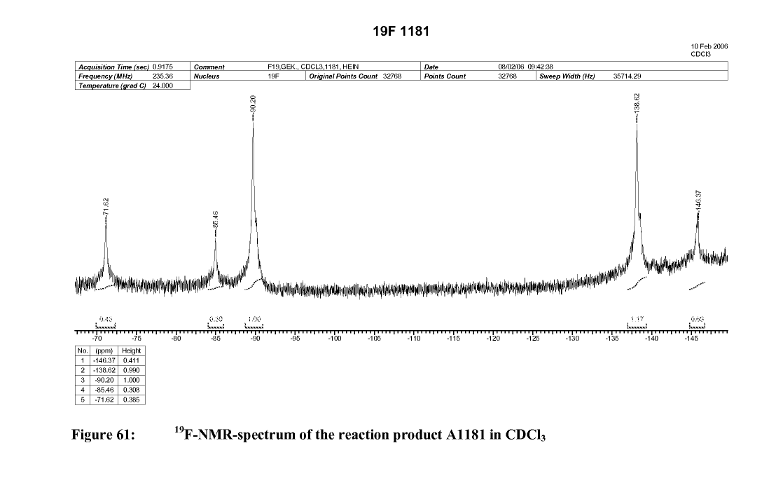

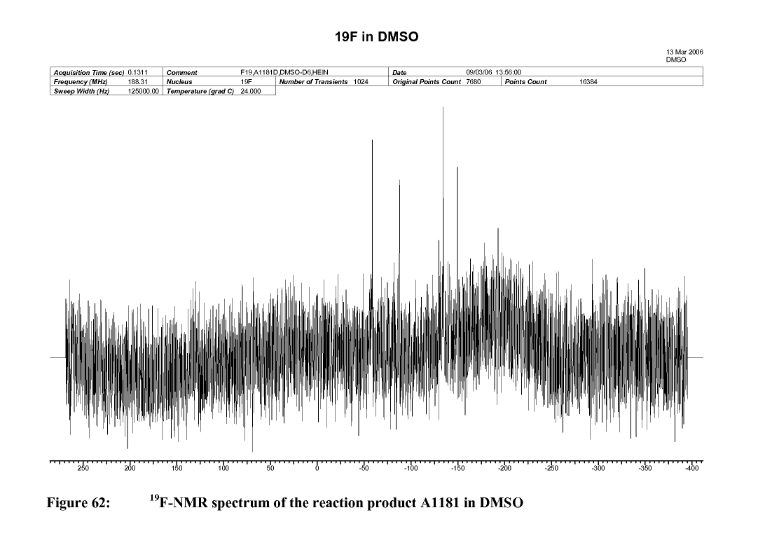

In FIG. 59 the ¹H-NMR-spectrum of the reaction product A1181 in CDCl₃ is shown, in FIG. 60 the ¹³C-NMR-spectrum of the reaction product A1181 in CDCl₃. In FIG. 61 the ¹⁹F-NMR-spectrum of the reaction product A1181 in CDCl₃ is shown, in FIG. 62 the ¹⁹F-NMR-spectrum of the reaction product A1181 in DMSO.

### Elemental Analysis

### Calculated onto 2 Groups

C₃₇H₂₀F₁₀N₂O₄S

778.62

778.098411

C, 57.08%; H, 2.59%; F, 24.40%; N, 3.60%; O, 8.22%; S, 4.12%

| | found | calculated |
|---|---|---|
| C | 51.82 | 57.08 |
| H | 3.07 | 2.59 |
| N | 2.31 | 3.60 |
| S | 4.44 | 4.12 |
| F | 15.93 | 24.40 |

### Referring to the Fluorine Content 1.31 Groups Per Repeat Unit are Bound!

### Cited Patent- and Non-Patentliterature

¹M. A. Hickner, H. Ghassemi, Y. S. Kim, B. R. Einsla, J. E. McGrath, Chem. Rev. 2004, 104, 4587-4612
²J. Kerres, W. Cui, S. Richie, J. Polym. Sci., Part A: Polym. Chem. 1996, 34, 2421
³J. Kerres, W. Zhang, W. Cui, J. Polym. Sci., Part A: Polym. Chem. 1996, 36, 1441
⁴J. Kerres, W. Cui, M. Junginger, J. Membr. Sci. 1998, 139, 227
⁵J. A. Kerres, A. J. van Zyl, J. Appl. Polym. Sci. 1999, 74, 428-438
⁶F. Wang, M. Hickner, Y. S. Kim, T. A. Zawodzinski, J. E. McGrath, J. Membr. Sci. 2002, 197, 231
⁷F. Wang, M. Hickner, Q. Ju, W. Harrison, J. Mecham, T. A. Zawodzinski, J. E. McGrath, Macromol. Symp. 2001, 175, 387
⁸A. Glüsen, D. Stolten, Membranen für Polymerelektrolyt-Brennstoffzellen, Chemie Ingenieur Technik 2003, 75 (11), 1591-1597
⁹H. R. Allcock, M. A. Hofmann, C. M. Ambler, R. V. Morford, Macromolecules 2002, 35, 3484
¹⁰K. Miyatake, A. S. Hay, Journal of polymer Science: Part A: Polymer Chemistry 2001, 39, 3770-3779
¹¹K. Jakoby, S. P. Nunes, K.-V. Peinemann, DE 101 48 131 A 1
¹²L. N. Markovsii, G. G. Furin, Y. G. Shermolovich, G. G. Yakobson, Bulletin of the Academy of Sciences of the USSR/Division of Chemical Sciences 1981, 30 (4), 646-648
¹³T. Yokomatsu, H. Abe, T. Yamagishi, K. Suemune, S. Shibuya, J. Org. Chem. 1999, 64, 8413-8418
¹⁴G. S. Cockerill, H. J. Easterfield, J. M. Percy, S. Pintat, J. Chem. Soc., Perkin. Trans. 1, 2000, 2591-2599
¹⁵J. T. Liu, H. J. Lu, Chin. Chem. Lett. Vol. 11, No. 3, pp 189-190, 2000
¹⁶G. K. Surya Prakash, J. Hu, J. Simon, D. R. Bellew, G. A. Olah, J. Fluorine Chem. 125 (2004) 595-601
¹⁷A. Taeger, C. Vogel, D. Lehmann, W. Lenk, K. Schlenstedt, J. Meier-Haack, Macromol. Symp. 2004, 210, 175-184
¹⁸L. Wang, Y. Z. Meng, S. J. Wang, X. Y. Shang, L. Li, A. S. Hay, Macromolecules 2004, 37, 3151-3158
¹⁹F. Liu, J. Ding, M. Li, M. Day, G. Robertson, M. Zhou, Macromol. Rapid Commun. 2002, 23, 844-848
²⁰Y. Qi, J. Ding, M. Day, J. Jiang, C. L. Callender, Chem. Mater. 2005, 17, 676-682
²¹J. Ding, F. Liu, M. Li, M. Day, P. Vuillaume, U.S. Provisional Patent Ser. 60/433,574 2003
²²A. N. Sergis, Reactions of sulphone-stabilised carbanions with fluorinated aromatic compounds, Ph. D. thesis, School of Chemistry, Thames Polytechnic London, UK, December 1990

1. Non-fluorinated, partly fluorinated or perfluorinated (preferentially non-, partly or perfluorinated) aromatic monomeric, oligomeric and polymeric sulfon-resp. phosphonic acid (resp. their derivatives), whereby the partly or perfluorinated groups of the polymer can be present as well in the main chain as well as in the side chain of the polymers, caracterised in that they are obtained by nucleophilic aromatic substitution with sulphur resp. phosphor nucleophiles.

2. Non-halogenated, partly halogenated or per halogenated (preferentially non-, partly or perfluorinated) aromatic monomeric sulfonic-resp. Phosphonic acids (resp. Their derivatives) according to claim 1, caracterised in that they contain a broad substitution pattern of reactive halogen aromates (preferentially fluoro aromates) and can carry proton conducting groups (see FIG. 1 and FIG. 3), whereby as halogenated monomers are preferred: bis(pentafluorophenyl)sulfone, bis(pentafluorophenyl)sulfide, decafluorobiphenyle, 4,4′-difluorobiphenyle, decafluorobenzophenone, 4,4′-difluorobenzophenone, bis(4-fluorphenyl)phenylphosphinoxide), decafluorodiphenylsulfide, hexafluorobenzole, pentafluorobenzole, different substituted di, tri- and tetrafluorobenzole, octafluorotoluene, 2,2′,3,3′,5,5′,6,6′-octafluorobiphenyle, pentafluoropyridine, different substituted di-, tri- and tetrafluoropyridine (e.g. 2,3,5,6-tetrafluoropyridine, 2,6-difluoropyridine, 3,5-difluoropyridine, 2,5-difluoropyridine, 2,4-difluoropyridine, 2,4,6-trifluoropyridine), different triazines (e.g. 2,4,6-trifluoro-1,3,5-triazine, 3,5,6-trifluoro-1,2,4-triazine, 3,6-difluor-1,2,4-triazine), pyrimidine (e.g. 2,4,6-trifluoropyrimidine), pyridazine (e.g. 3,6-difluoropyridazine, 3,4,5,6-tetrafluoropyridazine), pyrazine (e.g. 2,6-difluoropyrazine, 2,3,5,6-tetrafluoropyrazine), chinoline (e.g. heptafluorochinoline), isochinoline (e.g. heptafluoroisochinoline), quinoxaline (e.g. hexafluorquinoxaline), quinazoline (e.g. hexafluorquinazoline) as wel as non-, partly- or perfluorinated imidazoles and benzimidazoles or similar dihalogenated heteroaryl compounds, pentafluorobenzolsulfonic acid resp. their salts, pentafluorobenzolphosphonic acid resp. their salts and as diphenoles all possible diphenoles can be used, whereby the following diphenoles are preferred: bisphenol A (4,4′-(isopropylidene)-diphenole), bisphenol S (bis(4-hydroxyphenyl)sulfone), bis(4-hydroxyphenyl)thioether), bis(4-hydroxyphenyl)ether, 4,4′-(hexafluorisopropylidene)-diphenole, bis(4-hydroxyphenyl)phenylphosphinoxide and phenolphtalein, whereby the monomers can be combined in any way to polymers, e.g. to homopolymers, statistical copolymers or blockcopolymers.

3. Non-halogenated, partly halogenated or per halogenated (preferentially non-, partly or perfluorinated) aromatic oligomeric resp. polymeric sulfonic-resp. Phosphonicacids (resp. their derivatives) according to claim 1, caracterised in that they contain a broad substitution pattern of reactive halogen aromates (preferentially fluoro aromates) and can carry proton conducting groups (see FIG. 2 and FIG. 4), whereby all polymers, which contain an Cₛₚ_{²}-bound halogen (preferentially fluorine) are preferred, of which exemplarily some polymers are shown in the figures FIG. 6, FIG. 7, FIG. 8, FIG. 9, FIG. 10, FIG. 11, FIG. 12, FIG. 13, FIG. 14, FIG. 15, FIG. 16, FIG. 17, FIG. 18, FIG. 19, FIG. 20, FIG. 21, FIG. 22, FIG. 23, FIG. 24, FIG. 25.

4. Process to produce non-halogenated, partly halogenated or per halogenated (preferentially non-, partly or perfluorinated) aromatic monomeric, oligomeric and polymeric sulfonic-resp. phosphonic acids (resp. their derivatives) according to claim 1 to 3, characterised in that as solvent-depending on the substitution pattern of the educt-protic or dipolar-aprotic and aprotic solvents like water (only for the sulfonation), THF, diethylether, dioxane, glyme, diglyme, triglyme, DMAc, DMF, NMP, sulfolane, propylencarbonate, dimethylsulfoxide, acetonitrile, Benzene, Toluene, xylole as well as any mixtures of these solvent with each other can be used.

5. Process to produce non-halogenated, partly halogenated or per halogenated (preferentially non-, partly or perfluorinated) aromatic monomeric, oligomeric and polymeric sulfonic-resp. phosphonic acids (resp. their derivatives) according to claim 1 to 4, caracterised in that the reaction temperature is between −93° C. and +200° C. depending on solvent and reactivity of the educt and that the reactions are carried out in argon, nitrogen or in air.

6. Process to produce non-halogenated, partly halogenated or per halogenated (preferentially non-, partly or perfluorinated) aromatic monomeric, oligomeric and polymeric sulfonic-resp. phosphonic acids (resp. their derivatives) according to claim 1 to 5, characterised in that as reactive nucleophile is chosen a metalsulfite or metal hydrogensulfite (e.g. sodiumsulfite, sodiumhydrogensulfite, potassiumsulfite, potassiumhydrogensulfite) resp. a metal phosphite (e.g. sodiumdimethylphosphite, sodiumdiethylphosphite, sodiumdiphenylphosphite) or a posphitecompound like tris(trimethylsilyl)phosphite, that in a SNAr-reaction liberates one or more halogene ions (preferentially fluoride ions) from the corresponding partly- or perhalogenated (preferentially partly- or perfluorinated) starting compounds, whereby the metalphosphite is produced in situ by reaction from metal hydride and dialkyl- or diarylphosphite in THF or another water-free medium.

7. Process to produce non-halogenated, partly halogenated or per halogenated (preferentially non-, partly or perfluorinated) aromatic monomeric, oligomeric and polymeric sulfonic resp. phosphonic acids (resp. their derivatives) according to claim 1 bis 6, characterised in that in case of monomeric compounds a standard procedure (as described e.g. by Yakobson et al.¹²) or a corresponding work-up leads to the desired products, whereby the purification is a destillation for liquid products, a recrystallisation for solid products and in the case of polymeric (oligomeric) non-halogenated, partly halogenated or per halogenated (preferentially non-, partly or perfluorinated) aromatic sulfonic-resp. phosphonic acids (resp. their derivatives) the work-up and purification happens by repeated precipitation and dissolving and in the case of water-soluble polymeric sulfonic resp. phosphonic acids by dialysis.

8. Compounds, especially polymers and oligomers, produced by one or more of the processes of claims 1 to 7.

9. Use of compounds produced by one or more processes of claims 1 to 7 in membrane processes (especially fuel cells, membrane electrolysis and electro dialysis processes), coatings (e.g. textile fibers), nanoparticles, paints, glues, sealings, sensors, colours, herbicides und pesticides.
